SEQUENCE LISTING (8 sequences)

SEQ ID NO: 1 - DNA, 21 bp, synthetic construct
  source 1..21: mol_type = other DNA
cctgaggtccttttccaacca

SEQ ID NO: 2 - DNA, 20 bp, synthetic construct
  source 1..20: mol_type = other DNA
ggattccggcagcttccatt

SEQ ID NO: 3 - DNA, 22 bp, synthetic construct
  source 1..22: mol_type = other DNA
cattccgaggcttaatttaccg

SEQ ID NO: 4 - DNA, 22 bp, synthetic construct
  source 1..22: mol_type = other DNA
ctttcagttcccaacgaagacc

SEQ ID NO: 5 - DNA, 22 bp, synthetic construct
  source 1..22: mol_type = other DNA
ttggaattggtatttgcacctg

SEQ ID NO: 6 - DNA, 22 bp, synthetic construct
  source 1..22: mol_type = other DNA
tttttgctttgctctgatctgc

SEQ ID NO: 7 - DNA, 22 bp, synthetic construct
  source 1..22: mol_type = other DNA
ggtggaaaggtagctgcatgga

SEQ ID NO: 8 - DNA, 22 bp, synthetic construct
  source 1..22: mol_type = other DNA
tccatgcagctacctttccacc

(19) United States
(12) Patent Application Publication
(10) Pub. No.: US 2024/0067975 A1
(43) Pub. Date: Feb. 29, 2024

(54) Plastid-transgene-encoded nuclear gene silencing

(71) Applicant: Plastomics Inc., St. Louis, MO (US)

(72) Inventor: Jeffrey Staub, Wildwood, MO (US)

(21) Appl. No.: 18/142,777

(22) Filed: May 3, 2023

Related U.S. Application Data
(63) Continuation-in-part of application No. 18/108,996, filed on Feb. 13, 2023.
(60) Provisional application No. 63/309,804, filed on Feb. 14, 2022.
(60) Provisional application No. 63/351,575, filed on Jun. 13, 2022.

(51) Int. Cl. C12N 15/82

(52) U.S. Cl. CPC C12N 15/8214; C12N 15/8218

(57) ABSTRACT

A method for controlling expression of a nuclear-encoded gene in a plant comprising expressing a chloroplast-encoded dsRNA that silences an endogenous nuclear-encoded gene in the plant to produce a transformed plant line expressing the selected trait.

### CROSS-REFERENCE TO RELATED APPLICATIONS

This application is a continuation in part application of U.S. patent application Ser. No. 18/108,996 filed on Feb. 13, 2023, which claims priority to and the benefit of U.S. provisional patent application Ser. No. 63/309,804, filed on Feb. 14, 2022, and U.S. provisional patent application Ser. No. 63/351,575 filed on Jun. 13, 2022, the contents of each incorporated herein in their entireties.

### INCORPORATION OF SEQUENCE LISTING

The sequence listing that is contained in the file named “Sequence_Listing_P1810003US2.xml,” which is 8 kilobytes as measured in Microsoft Windows operating system and was created on Aug. 17, 2023, and is filed electronically herewith and incorporated herein by reference.

### FIELD OF THE INVENTION

The present invention relates to methods of knockdown of nuclear-encoded genes by way of plastid-encoded transgenes and more specifically to transplastomic plant lines wherein a plastid-transgene-encoded RNA exerts a gene silencing effect on nuclear-encoded gene.

### BACKGROUND

Plastid transformation technology was first developed in the model crop, Nicotiana tabacum, nearly 30 years ago (Svab et al., 1990; Svab and Maliga, 1993). Plastid transformation is an attractive technology for potential commercialization of crops engineered with biotechnology traits for several reasons: introduction of traits into the plastid genome via an active homologous recombination system facilitates trait gene stacking, the possibility for high level transgene expression, especially in the abundant chloroplasts of leaves, and natural transgene containment due to maternal inheritance of plastids and lack of outcrossing via pollen in most crop plants (Maliga, 2004; Bock, 2013; Greiner et al., 2015; Maliga and Bock, 2011)). The technology has been used to study numerous facets of plastid biology and for expression of transgenes that impart useful traits, such as insect control, herbicide tolerance and introduction of new metabolic pathways to enhance nutritional (Ye et al., 2001; Dufourmantel et al., 2007; Zhang et al., 2017; Bally et al., 2018; Apel and Bock, 2009; Staub et al., 2000). Plastid transformation has been reported in numerous plants species, including commercial row crops like soybean (Dufourmantel et al., 2004), though is currently routine only in multiple Solanaciae species.

Among the numerous trait concepts tested using plastid transformation technology, transkingdom RNAi (TK-RNAi) has recently shown great promise for controlling some insect pests (Zhang et al., 2017; Bally et al. 2018). TK-RNAi utilizes the production in a transgenic plant of long double-stranded RNA (dsRNA), long self-complimentary hairpin RNA (hpRNA) or small interfering RNA (siRNA) derived from an essential gene of a plant pest to direct the pest's own RNAi machinery to degrade the target mRNA upon herbivory or infection. In plastid transgenics (transplastomic plants), expression of high levels of dsRNA or hpRNA was effective against Coleopteran (Zhang et al., 2015) and Lepidopteran (Jin et al., 2015; Bally et al., 2016) insect pests. Although Coleopterans are relatively susceptible to RNAi mediated by dsRNA longer than ˜60 bp, most Lepidopteran insects can be difficult to control, due in part to extracellular RNAses in their digestive tract and haemolymph that degrades the dsRNA prior to initiation of a potent RNAi response. Expression in chloroplasts of 189 nucleotide (Bally et al. 2016) or ˜270 nucleotide (Jin et al., 2015) hpRNA vectors sequestered in chloroplasts was suggested to help provide efficacy against recalcitrant Helicoverpa insects. In each of these reports, no small RNA production from plastid transgenes was observed, leading the authors to conclude that Dicer and the rest of the RNAi machinery is absent from plastids (Zhang et al., 2015; Bally et al. 2016; Zhang et al. 2017; Bally et al. 2018). While plastid produced long dsRNA and hpRNA is effective against some insect pests, the lack of small RNA production in transplastomic plants may be a limitation for TK-RNAi in controlling pathogens that take up small RNAs, for example, fungi (Niu et al., 2021; Qiao et al., 2021), aphids (Sattar and Thompson, 2016; Biedenkopf et al., 2020), plant viruses (Gaffar and Koch 2019) and nematodes (Medina et al., 2017; Tian et al., 2019).

Currently, another obvious limitation of plastid engineering is that the plastid transgene function is sequestered to the organelle. Thus, nuclear-encoded genes whose function resides outside of the organelle, including metabolic pathways in the cytoplasm or regulatory functions in the nucleus, have not been amenable to plastid engineering. Although plastids import up to ˜3000 nuclear-encoded proteins (Sjuts et al., 2017; Paila et al., 2015), there is no known mechanism for plastid-encoded gene products to function outside of the organelle. Evidence suggests, however, that plastid-encoded proteins and RNAs can be found in other cellular compartments. For example, during the natural process of chloroplast autophagy, Rubisco and other chloroplast-encoded proteins can be observed in transit to or in vacuoles prior to protein degradation (Otegui, 2017; Izumi et al., 2015). While plastid DNA can “escape” to the nucleus, intact plastid-encoded RNAs have not been observed outside of the organelle, though plastid-encoded tRNA fragments have been observed in the cytoplasm and suggested to be involved in regulatory processes (Alves and Nogueira, 2021). Over evolutionary time frames, plastid genes have migrated to the nucleus (McFadden, 2001), and can “escape” during plastid or mitochondrial transformation experiments when strong selection is used to identify rare transfer of DNA from the organelle to the nucleus (Wang et al., 2018; Fuentes et al., 2012; Stegemann and Bock, 2006; Stegemann et al., 2003; Thorsness and Fox, 1990). These observations suggest a means by which plastid DNA or RNA can exit the organelle to function in other cellular compartments.

### SUMMARY

An aspect of the present disclosure relates to a novel finding that a plastid-encoded double-strand RNAi gene or sense-strand RNA gene can catalyze silencing of an endogenous nuclear gene to create a (useful or beneficial) trait in a photosynthetic plant. It is otherwise previously unknown that a plastid-encoded gene could affect functions outside of the organelle. It has never before been observed that any dsRNAi transgene expressed in plastids could unexpectedly produce classical “small RNAs” (21-24 nucleotides) that enter the nuclear gene silencing pathway. All dsRNAi constructs that have been expressed in chloroplasts prior to the instant disclosure have been thought not to be processed into smaller RNAs, which is an advantage for “transkingdom RNAi” against some pathogens (i.e., fungi, bacteria, plant viruses and some insects) that are known to be susceptible to long dsRNAi constructs. Aspects of this disclosure thus relate to the processing of chloroplast dsRNAi into small RNAs such as 21 to 24 nucleotides in length which will also enable transkingdom RNAi against other important pathogens that are susceptible to those small RNAs, such as other fungal, bacterial, viral and some insect pathogen. Furthermore, plastid-expressed dsRNAs efficiently knockdown mRNA expression a plant nuclear-encoded gene, as exemplified by knockdown of the nuclear-encoded phytoene desaturase gene (PDS) and plastid-expressed sense-strand RNAs also reduce PDS activity but with no change to PDS mRNA levels, suggesting another mechanism of action of plastid-expressed RNAs and potentially providing a simple method to affect the expression of nuclear-encoded plant genes.

Another aspect of the present disclosure relates to a method of introducing a DNA construct into a plant plastid genome to silence an endogenous nuclear-encoded gene to impart a beneficial trait to the plant.

The expression of a plastid-encoded dsRNA silences the endogenous nuclear-encoded gene.

In one or more embodiments, the method includes introducing a plurality of DNA constructs into a plant plastid genome to silence one or more endogenous nuclear-encoded genes to impart one or more beneficial traits to the plant.

In one or more embodiments, the plastid-encoded dsRNA enables accumulation of small RNAs capable of gene silencing.

In one or more embodiments, the small RNAs have a length in the range of 21 to 24 nucleotides.

The small RNAs accumulated are 21 or 24 nucleotide small RNAs capable of gene silencing.

In one or more embodiments, the method comprises using convergent plastid promoters or hairpin stem/loop dsRNA constructs for creating the plastid-encoded dsRNA that produces 21 nucleotide small RNAs.

The expression of a plastid-encoded sense-strand RNA construct silences the activity of an endogenous nuclear-encoded gene.

Yet another aspect of the present disclosure relates to a plant cell having a plastid-encoded dsRNA that enables accumulation of 21-24 nucleotide small RNAs capable of silencing a nuclear-encoded gene for imparting a selected trait to a photosynthetic plant.

Convergent plastid promoters or hairpin stem/loop dsRNA constructs created the plastid-encoded dsRNA that produces the 21-24 nucleotide small RNAs.

The dsRNA enters a host cytoplasmic RNAi pathway to generate the 21-24 nucleotide small RNA independent of host homologous sequences.

Another aspect of the present disclosure relates to a method for controlling expression of a nuclear-encoded gene in a plant comprising expressing a plastid-encoded dsRNA that silences an endogenous nuclear-encoded gene in the plant to produce a transformed plant line expressing the selected trait.

Selecting a recipient plant cell, transforming the plant cell by introduction of a plastid transformation vector effecting expression of the plastid-encoded dsRNA capable of silencing the nuclear-encoded gene to the plant cell, and regenerating the plant from cells expressing the selected trait.

Another aspect of the present disclosure relates to a method for controlling expression of a nuclear-encoded gene in a plant comprising expressing a plastid-encoded sense-strand RNA construct that silences activity of an endogenous nuclear-encoded gene in the plant to produce a transformed plant line expressing the selected trait.

The method further comprises selecting a recipient plant cell, transforming the plant cell by introduction of a plastid transformation vector effecting expression of the plastid-encoded sense-strand RNA construct capable of silencing activity of a nuclear-encoded gene to the plant cell, and regenerating the plant from cells expressing the selected trait.

Yet another aspect of the present disclosure relates to a method for controlling a plant pathogen that is susceptible to 21-24 nucleotide small RNAs via expression of a plastid encoded dsRNA that silences an essential gene in the pathogen.

The method further includes selecting a recipient plant cell, transforming the plant cell by introduction of a chloroplast transformation vector effecting expression of the plastid encoded dsRNA capable of silencing the nuclear-encoded gene to the plant cell, and regenerating the plant from cells expressing the selected trait.

Yet another aspect of the present disclosure relates to a transformed photosynthetic plant cell expressing a plastid-encoded dsRNA that silences an endogenous nuclear encoded gene wherein the plant cell exhibits a selected non-naturally occurring trait.

A transformed photosynthetic plant cell expressing a plastid-encoded sense-strand RNA construct that silences the activity of an endogenous nuclear encoded gene wherein the plant cell exhibits a selected non-naturally occurring trait.

Another aspect of the present disclosure relates to a method of introducing a plurality of DNA constructs into a plant plastid genome to silence one or more endogenous nuclear-encoded genes, one or more pathogens, or a combination thereof to impart one or more beneficial traits to the plant.

The plastid-encoded dsRNA enables accumulation of small RNAs which enable transkingdom RNAi against the one or more pathogens.

Yet another aspect of the present disclosure relates to a transformed photosynthetic plant cell having a plastid-encoded dsRNA that enables accumulation of small RNAs capable of silencing a nuclear-encoded gene for imparting a selected trait to the plant.

The small RNAs may be 24 nucleotide small RNAs.

In any one or more of the embodiments above the plant cell or plant plastid genome is a monocot or dicot plant cell or plant plastid genome.

In any one or more of the embodiments above the plant is a soybean plant.

In any one or more of the embodiments above, the plant is a tobacco plant.

Yet another aspect of the present disclosure relates to a method of liberating a nucleic acid or protein from a transgenic plant plastid to function in a non-plastid compartment of the cell and a treatment that enhances the liberation or escape of a transgene-encoded RNA or protein from transgenic plastids, including treatment of the plants with a chemical herbicide, antimicrobial or chemical agent, or biotic stress conditions that affects plastid membranes.

The treatment is performed in tissue culture or via application to the plant growing in soil and further comprising titrating the chemical herbicide, antimicrobial or chemical agent to enable recovery of the plant after treatment.

### BRIEF DESCRIPTION OF THE DRAWINGS

FIG. 1. Plastid-expressed sense and double-strand RNA PDS transgenes produce pigment deficient tobacco seedlings. FIG. 1A illustrates PDS sense RNA (PTS38), antisense RNA (PTS39) and dsRNA (PTS40) transgenes and the aadA selectable marker are integrated into the plastid genome between the resident trnG and trnfM plastid genes via homologous flanking regions. Oligonucleotide probes used for PDS gene detection are shown by the red lines. FIGS. 1B and 1C show 12-day old T1 seedlings derived from self-fertilized TO plants are sown on media lacking antibiotics (FIG. 1B) or containing spectinomycin (FIG. 1C). Cr., Chlamydomonas reinhardtii.

FIG. 2. Maternal inheritance of plastid-encoded pigment deficiency are shown in FIGS. 2A and 2B where T1 seedlings derived from reciprocal crosses of TO transplastomic plants to wildtype plants were (e.g. WT(♀)×PSV38(♂)) sown in the absence (FIG. 2A) or presence (FIG. 2B) of spectinomycin antibiotic. In the case of PTS38 and PTS40 lines, pigment deficiency is observed only when those lines are used as female in crosses to wild-type plants used as the male, indicating the pigment deficiency is maternally inherited and plastid-encoded. FIG. 2C shows while cotyledons are completely bleached in the PTS38 and PTS40 seedlings, the first true leaves become green (red arrows). FIG. 2D shows T1 green plants placed in soil show rapid bleaching of new leaves. FIG. 2E is confocal images showing size, development stage and location of plastids in mesophyll and epidermis cells in leaves of wild-type, PTS38 and PTS40 lines. The scale bar equals 20 μm. ♀, female; ♂, male.

FIG. 3: Abundance and expression pattern of nuclear- and plastid-encoded PDS genes. Relative abundance of nuclear-encoded PDS1 and PDS2, as measured by qRT-PCR, in 12-day-old (FIG. 3A) and 24-day-old (FIG. 3B) seedlings from wild-type and PTS38, PTS39 and PTS40 transplastomic lines. All RNA expression values are relative to wild-type and normalized to the housekeeping gene, Actin. Error bars represent standard error around the mean. *, **, *** denotes p-value <0.05, 0.01, 0.001, respectively, Student's t-test. FIG. 3C is a Northern blot with wild-type and independent transplastomic events of PTS38, PTS39 and PTS40 lines. Membrane was either blotted with a probe recognizing the sense strand of PDS1 (left) or antisense strand of PDS1 (right). Ladder is RiboRuler High Range RNA ladder. Gel image before transfer is represented below the blot. Note that transcripts for the endogenous nuclear-encoded PDS1 and PDS2 genes are expected to be expressed at much lower levels than the plastid transgenes and are not observed on the northern blot.

FIG. 4. Plastid-expressed dsRNA is processed to 21-nt phasiRNAs as shown in FIGS. 4A and 4B mapping and accumulation of PDS1 siRNAs (red, sense strand; blue, antisense strand) in the PTS38 and PTS40 transplastomic lines. (FIG. 4A) Accumulation of siRNAs of predominantly 21-nt (insert) accumulate on both strands of PDS1 in the PTS40 lines (FIG. 4B) whereas siRNAs in the PTS38 line map to the PDS1 sense strand and show no bias in length (insert). The relative location of the PDS transgene fragment (grey) in the nuclear-encoded PDS1 gene (light blue) are represented above the panels. At FIGS. 4C and 4D, mapping and accumulation of siRNAs in the MT90 and MT94 lines expressing the Frankliniella occidentalis Actin-5c gene from either convergent promoters (FIG. 4C) or a hairpin/loop RNA (FIG. 4D) transgene are shown. Inserts show the length distribution of reads showing predominantly 21 nt siRNAs. The relative location of the transgene fragment (grey) in the Actin-5C gene (light blue) are represented above the panels. The y-axis indicates the distribution of read abundance normalized in reads per million (RPM).

FIG. 5. Nuclear-encoded PDS genes organization, alignment and fragment insertion into the plastid genome. FIG. 5A shows intron (blue lines) and exon (blue box) structure of the two nuclear-encoded PDS genes and cDNA fragment (red boxes) used for insertion into the plastid genome. FIG. 5B shows alignment of PDS1, PDS2 and the PTS transgene cDNA fragment. A 13 nucleotide sequence was inserted in the transgene fragment to discriminate plastid transgenes from the nuclear PDS genes. FIG. 5C is a Southern blot showing the integration of the PDS1 fragment and aadA transgenes at the expected location in the plastid genome. BglII restriction enzyme digest results in a ˜5.8 kb fragment in the transplastomic lines while wild-type plants without transgene insertion carry an ˜3.5 kb band. Note lack of the ˜3.5 kb band in transplastomic lines indicating they are homoplasmic for transgenes insertion.

FIG. 6. Mapping and abundance of PDS siRNAs in the PTS39-3 antisense RNA line. Low abundance siRNAs accumulate on both sense and antisense strands in the PTS39-3 line. Although abundance is low, the predominant read length is 21 nt (insert) and a moderate phasing score is observed for these siRNAs. The relative location of the transgene fragment (grey) in the PDS gene (light blue) are represented above the panels. The y-axis indicates the distribution of read abundance normalized in reads per million (RPM)

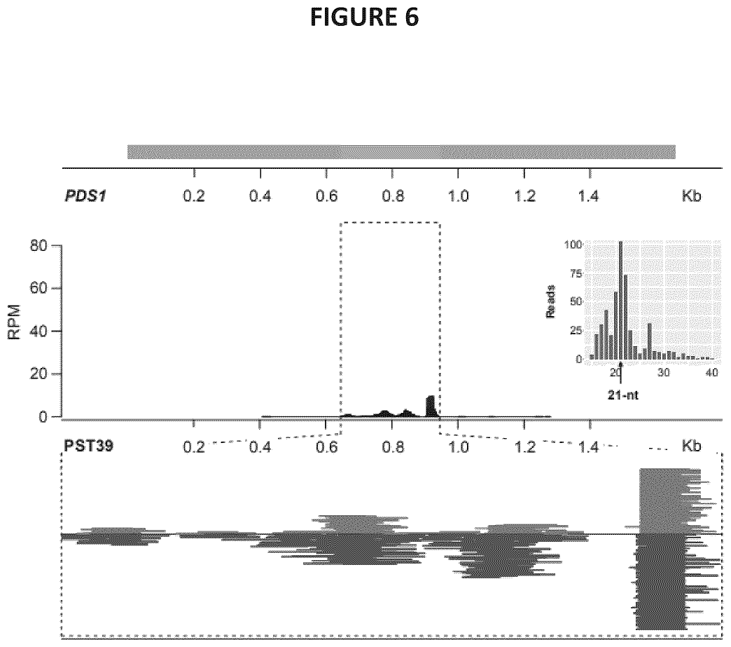

FIG. 7. siRNAs in the PTS38 and PTS40 lines do not map to the PDS2 nuclear gene polymorphic regions. The figure shows the location of nucleotide polymorphisms (orange bars) between PDS1 (left) and PDS2 (right) gene regions. SiRNA reads distributed to each gene are shown for PTS38 (middle) and PTS40 (bottom) lines. Note the absence of siRNAs mapping to the 7 polymorphic positions of the PDS2 gene. The y-axis indicates the distribution of read abundance normalized in reads per million (RPM).

FIG. 8. Mapping of transgene derived small RNAs in purified chloroplast fractions of PTS38 and PTS40 lines. Mapping and accumulation of PDS1 small RNAs (red, sense strand; blue, antisense strand) in the chloroplast fractions of PTS38 at FIG. 8A and PTS40 at FIG. 8B transplastomic lines. The relative location of the PDS transgene fragment (grey) in the nuclear-encoded PDS1 gene (light blue) is represented above the panels. Note that the mapping pattern of small RNA reads from the PTS40 transgene differs from the whole-cell RNA fraction because sRNAs accumulate only on the positive strand of the transgene, and neither line accumulates any phasiRNAs in the chloroplast fraction. The y-axis indicates the distribution of read abundance normalized in read per millions (RPM).

FIG. 9. Shows that plastid-expressed dsRNA is processed to 21-nt phasiRNAs in the absence of a nuclear-encoded mRNA target. (A-B) Mapping and accumulation of siRNAs in the MT91 and MT95 lines expressing the insect (Frankliniella occidentalis) SN7F gene from either convergent promoters in FIG. 9A or a hairpin/loop RNA in FIG. 9B transgene. Inserts show the length distribution of reads showing predominantly 21 nt siRNAs. The relative location of the transgene fragment in the SNF7 gene is represented above the panels. The y-axis indicates the distribution of read abundance normalized in reads per million (RPM).

FIG. 10 shows results of the PCR assay of each purified cellular fraction for the presence of chloroplast transcripts (16S rRNA), nuclear gene transcripts (PDS2) or mitochondrial transcripts (Mp002). Results show that purified plastid fractions (top gel) were not contaminated with nuclear (middle gel) or mitochondrial transcripts (bottom gel). Note that whole cell extracts carry nuclear (middle gel), plastid (top gel) and mitochondrial transcripts (bottom gel).

FIG. 11 illustrates cDNA sequences from F. virguliforme (Fvi) and S. sclerotiorum (Ssc) expressed from convergent plastid promoters (Pr) to create dsRNA constructs. Selection of plastid transformants is via a selectable marker (SM) cloned next to the dsRNA transgene and surrounding by cloned plastid DNA regions to direct homologous recombination and insertion into the soybean plastid genome.

### DETAILED DESCRIPTION

The present disclosure relates to a showing that plastid-encoded transcripts can leave the organelle and function in the cytoplasm to generate small RNAs that silence plant nuclear genes. The ability to silence plant nuclear genes from the plastid now enables crop improvement via engineering a vast array of host metabolic and other processes (reviewed in Mansoor et al., 2006; Saurabh et al., 2014), including biotic and abiotic stress (Rajput et al., 2021), and biomass (Feldmann, 2006) and grain yield (Feldmann, 2006; Shomura et al., 2008) with the advantages that plastid transformation brings of gene containment, lack of gene silencing and easy breeding via maternal inheritance.

In addition to plastid-encoded double-strand RNA-mediated gene silencing, the present disclosure also relates to a showing that a plastid-encoded sense-strand transcript can silence an endogenous plant nuclear gene. This unexpected result further expands the utility of plastid-encoded transgenes and may simplify some aspects of nuclear gene silencing using this approach.

A study was undertaken to test whether plastid-expressed transgenic RNAs can escape the organelle and function in the cytoplasmic post-transcriptional gene silencing pathway. One non-limiting example in the study is directed to Tobacco (Nicotiana tabacum) plastid transformants created that express a fragment of the nuclear-encoded Phytoene desaturase (PDS) gene, for which knockdown or translational inhibition of its cytoplasmic-localized mRNA results in an easily discernible, pigment deficient phenotype (Senthil-Kumar et al., 2007; Busch et al., 2002). dsRNA, sense, and antisense transcripts were expressed against PDS in transplastomic tobacco and multiple lines of evidence that the plastid-encoded transgenes affect nuclear PDS gene silencing were found. Highly efficient dsRNA-mediated knockdown of PDS was confirmed by qRT-PCR and small RNA sequencing, which indicated processing of the plastid-expressed dsRNA into 21-nt phasiRNAs, giving direct evidence for a post-transcriptional gene silencing (PTGS) mechanism. Interestingly, transplastomic lines that express dsRNA against insect gene targets, for which no pairing partner is encoded in the nuclear genome of tobacco, also process their long dsRNAs into 21-nt phasiRNAs. Further, plastid-expressed sense-strand RNAs efficiently knockdown PDS activity, but with no change to PDS mRNA levels, suggesting, without being bound by theory, another mechanism of action of plastid-expressed RNAs. The results indicate a common process of RNA escape from plastids to the cytoplasm that can be exploited for knockdown of host nuclear-encoded genes and expands the repertoire of biotechnology tools afforded by plastid transformation technology. Furthermore, the processing of chloroplast dsRNAi into small RNAs such as 21 to 24 nucleotides in length which will also enable transkingdom RNAi against other important pathogens that are susceptible to those small RNAs, such as other fungal, bacterial, viral and some insect pathogen

The present disclosure also relates to a method for directing plastid-encoded dsRNA against a nuclear-encoded host gene resulting in knockdown of the host mRNA in the cytoplasm is disclosed herein. As a model, the knockdown of the nuclear-encoded phytoene desaturase (PDS) gene, often used as a model for RNAi due to the easily discernible phenotype of bleached leaves that results from PDS knockdown, was tested. Surprisingly, dsRNA and sense-strand mediated knockdown of the nuclear-encoded PDS from plastid-encoded transgenes was observed. Furthermore, the plastid-encoded dsRNA is processed to small RNAs in the cytoplasm, indicating that the chloroplast-encoded dsRNA enters the cytoplasm gene silencing pathway.

Testing whether plastid-encoded RNA can escape the plastid and function outside of the organelle is also discussed throughout this disclosure. In further detail, methods were used to express in tobacco (Nicotiana tabacum) plastids, dsRNA, sense and antisense transcripts against phytoene desaturase (PDS), a small nuclear-encoded gene family whose knockdown of its cytoplasmic-localized mRNA results in an easily discernible pigment deficient phenotype. As mentioned briefly above, a highly efficient plastid-encoded sense-strand RNA and dsRNA-mediated knockdown of the nuclear-encoded PDS genes was observed. The small RNA (sRNA) sequencing analyses provides evidence for nuclear gene silencing via processing of plastid-transgene-derived transcripts in the cytoplasm into 21-nt phased-short interfering RNA (phasiRNA). Furthermore, transplastomic lines that express dsRNA against insect gene targets, for which no pairing partner is encoded in the nuclear genome of tobacco, also process their long dsRNAs into 21-nt phasiRNAs, indicating that processing of plastid-expressed dsRNA into 21 nt phasiRNAs occurs broadly. Interestingly, plastid-expressed sense-strand RNAs efficiently knockdown PDS activity but with no change to PDS mRNA levels, suggesting another mechanism of action of plastid-expressed RNAs.

The methods, materials, and results are discussed in further detail below and in the accompanying figures.

Construction of Plastid Transformation Vectors

PDS fragments in pPTS40 and pPTS41 were synthesized and cloned as KpnI/SbfI DNA fragments between the convergent Prrn promoters in vector pJZ199 (Zhang et al., 2015). In pPTS38, the E. coli rrnB terminator fragment was synthesized and cloned as a KpnI/NotI fragment to replace one of the Prrn promoters, to create the sense PDS construct. In pPTS39, the rrnB terminator was cloned as an SbfI/SalI fragment to replace the opposite Prrn promoter, creating the antisense PDS construct. The PDS transgenes are located next to a chimeric aadA spectinomycin resistance gene driven by Chlamydomonas reinhardtii chloroplast psbA gene promoter and rbcL gene 3′-untranslated region. The PDS and aadA transgenes are flanked by regions of identity (˜1950 nt and ˜670 nts) to the tobacco chloroplast genome, resulting in integration of both transgenes between the resident trnfM and trnG chloroplast genes in plastid transformed plants.

Plant Growth, Transformation and Selection of Chloroplast Transformants

Nicotiana tabacum cv Petit Havana plants are grown aseptically from seedlings on MS agar medium for ˜4 weeks at 28° C. in 16 hr light/8 hr dark cycling light (using 4500K cool fluorescent bulbs). Young leaves are harvested for particle bombardment, placed abaxial side up and bombarded using the BioRad PDS1000 He gun according to standard procedures (Maliga and Svab 2011). Transplastomic events are selected by growth on 500 mg/L spectinomycin. Primary transformants typically arise as shoots on this medium; young leaf tissue from shoots is dissected and used for a second and subsequently repeated for a third round of plant regeneration on selective medium to ensure homoplasmy of the plastid transformed lines. Plastid transformants are confirmed by PCR and PCR-sequencing of amplification products to confirm transgene insertion and identity in each transformed line.

Plastid transformed lines are rooting on MS medium containing 500 mg/L spectinomycin and allowed to grow to the 4-5 leaf stage before transfer to soil and maturity in the greenhouse. For reciprocal crosses of plastid transformed lines to wild-type plants, flowers are emasculated by hand and manually pollinated, then individually bagged until seed pods are mature. Seeds are surface sterilized using 10% Chlorox solution with a few drops of Tween-20 for 10 minutes with shaking. After sterilization, seeds are washed with at least four changes of excess sterile water. Sterilized seeds are sown on MS agar medium.

Southern Blot Analysis of Plastid Transformed Lines

Leaves from plants or seedlings grown aseptically in tissue culture are used for total cellular DNA isolation using the DNAZol reagent (ThermoFisher) according to manufacturer's instructions. 3 ug of total cellular DNA is digested by BglII restriction enzyme, electrophoresed in an agarose gel, and digested DNA transferred to nylon membrane according to standard procedures. The probe for DNA hybridization is synthesized using DIG probe kit (Sigma-Aldrich) and used for overnight hybridization at 55° C. Washing and processing of the blot is performed according to standard procedures.

Whole-Cell and Chloroplast-Enriched RNA Fraction Isolation, Library Construction, and Sequencing

Seeds from chloroplast-transformed T1 plants were sown in petri dishes under controlled environment at 28° C. with 16 hours of light. Plantlets were grown for 12-24 days prior to harvest (pPTS38, pPTS39 and pPTS40 constructs plus wildtype) except for plants of construct pPTS41 that were transferred and grown in the greenhouse for several weeks. A total of two events per construct were grown for isolation of whole-cell and chloroplast-enriched RNA fractions. To collect the whole-cell RNA fraction, plantlets were harvested in three or five replicates and, after tissue dissection, samples were immediately frozen in liquid nitrogen and kept at −80° C. before RNA isolation. To isolate the chloroplast-enriched RNA fraction, we isolated chloroplasts from fresh leaves using the Minute™ Chloroplast Isolation Kit (Invent Biotechnologies, Inc. Plymouth, MN, USA), following the manufacturer's instructions. In order to isolate enough RNA, per sample, chloroplast was isolated from two preps of 250 mg of tobacco leaf. The whole-cell and chloroplast-enriched RNA fraction were isolated with the TRI Reagent (Sigma-Aldrich, St. Louis, MO, USA) following the manufacturer's instructions.

Determining the Purity of Isolated Chloroplasts

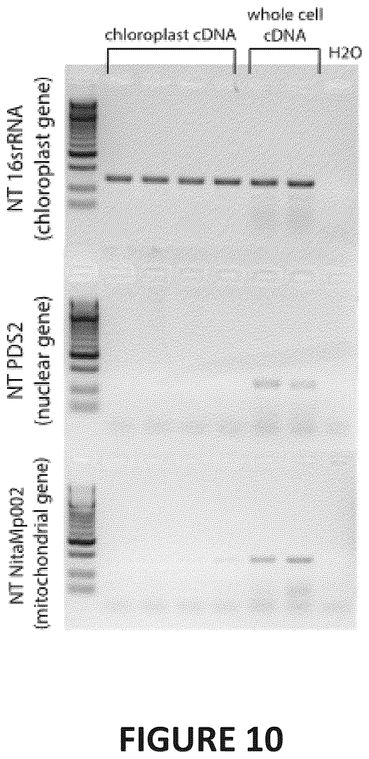

cDNA was generated from whole cell RNA fractions and from purified plastid fractions using the TetroRT kit (Meridian Bioscience, Cincinnati, OH, USA), using Random Hexamer priming to synthesize the cDNA rather oligo-dT. Each cellular fraction was assayed for the presence of a chloroplast transcripts (16S rRNA), nuclear gene transcripts (PDS2) or mitochondrial transcripts (Mp002). As can be seen in the results in FIG. 10, purified plastid fractions (top gel) were not contaminated with nuclear (middle gel) or mitochondrial transcripts (bottom gel). As expected, whole cell extracts carry nuclear (middle gel), plastid (top gel) and mitochondrial transcripts (bottom gel).

To further assess the purity of chloroplast-enriched RNA, between the whole-cell and chloroplast-enriched RNA fractions, we compared the abundance of two highly expressed miRNAs in tobacco leaves and observed that chloroplast RNA is enriched at 90%. (Table 1).

TABLE 1
Purity of isolated chloroplast fractions as estimated
from abundance of contaminating nuclear-encoded miRNAs.

| Abundance of miR156 | | | | Abundance of miR168 | | |
|---|---|---|---|---|---|---|
| Construct | | Whole- | | | Whole- | |
| name | Chloroplast | cell | CHL/WCE | Chloroplast | cell | CHL/WCE |
| PTS38-4 | 889.5 | 12,950.3 | 0.07 | 325.3 | 4505.7 | 0.07 |
| PTS38-8 | 1197.6 | 12,151.3 | 0.10 | 312.6 | 3260.2 | 0.10 |
| PTS39-3 | 1285.5 | 23,302.2 | 0.06 | 307.6 | 4592.3 | 0.07 |
| PTS39-4 | 802.0 | 22,191.6 | 0.04 | 212.7 | 5688.2 | 0.04 |
| PTS40-4 | 418.9 | 13,439.8 | 0.03 | 194.6 | 4585.8 | 0.04 |
| PTS40-6 | 439.0 | 13,010.1 | 0.03 | 163.7 | 4177.1 | 0.04 |
| WT | 609.8 | 18,536.0 | 0.03 | 147.0 | 4367.5 | 0.03 |

sRNA libraries were constructed using the RealSeq-AC miRNA Library Kit for Illumina sequencing (Somagenics, Santa Cruz, CA, USA) using an input of 150 ng total RNA and 16 PCR amplification cycles. sRNA libraries were size-selected for the end product of ˜150-nt using the SPRIselect Reagent (Beckman Coulter Life Sciences, Indianapolis, IN USA) magnetic beads. Overall, a total of 69 sRNA libraries were constructed. All libraries were quantified on a DeNovix apparatus (Wilmington, DE USA) using the Qubit dsDNA Assay Kit (Thermo Fisher Scientific, Waltham, MA USA) and libraries were multiplexed in 10 nM pools. Single-end sequencing was performed with 76 cycles (3 lanes). The sequencing was generated on an Illumina NextSeq 550 instrument (Illumina) at the University of Delaware DNA Sequencing and Genotyping Center.

Bioinformatics Analysis and Visualization of sRNA-Seq Data

Cutadapt v3.4 (Martin, 2011) was used to preprocess sRNA-seq reads, removing the 3′ adapter and discarding trimmed reads shorter than 15-nt or longer to 40-nt. Cleaned reads were mapped to PDS1 (NCBI ID: XM_016610712.1) and PDS2 (NCBI ID: XM_016642615.1) transcripts using ShortStack v3.8.5 (Johnson et al., 2016) with the following parameters: -mismatches 0, -mmap u, -dicermin 15, -dicermax 40, and -mincov 1.0 reads per million (RPM). We used the ShortStack analysis report to identify a phasiRNA-generating feature over reads mapping each PDS gene for each study. A “Phase Score” exceeding or equal to 20 was considered as true positive phasiRNA.

To visualize sRNA mapping over PDS genes, mapping files were converted into Bed Graph and Bed files using functions genomecov and bamtobed from bedtools v2.30.0 (Quinlan and Hall, 2010), respectively. The R package Sushi v1.24.0 (Phanstiel et al., 2014) was used to represent the position of transgenes over PDS genes and to visualize the coverage and read distribution of sRNAs over PDS1 and PDS2 genes.

To investigate properties of sRNA mapping to PDS genes, the (i) read length distribution, (ii) the nucleotide composition of reads, and (iii) the distribution of reads over PDS genes for constructs with a significant Phase Score were analyzed. (i) First, summarizing sRNA reads mapping to PDS genes into unique tags. The total number of reads per length were counted and the R ggpubr package (Wickham, 2016; Kassambara, 2020) was used to draw a bar plot of the read length distribution. (ii) Second, investigating the nucleotide composition. The frequency of nucleotides at each position was investigated and the R ggpubr package used to visualize these frequencies. (iii) For constructs with a significant Phase Score, assigning the positions within the PDS genes to “phasing” bins to one of the 21 arbitrary bins, which repeat in 21-nt cycles (lines) was done. The abundance of reads in each bin was calculated and visualized results on a radar plot. The method described in the chapter “A Method to Discover Phased siRNA Loci” (Meyers and Green, 2009) was followed.

RT-PCR Validation of PDS Knockdown in Plastid Transformed Lines

Without being bound by theory, the expectation is that the chloroplast PCR primers will amplify in both the whole cell extract and the isolated chloroplasts, while the nuclear control genes will only amplify in the whole cell extract. This proves that the inventors can do chloroplast isolations and provides a basis for RT-PCR as a method to analyze these tobacco samples.

qRT-PCR of PDS1 and PDS2 Levels in Transgenic Plants

To examine levels of PDS1 and PDS2 in PTS38, 39, and 40, seedlings were collected 12 days and 4 weeks after sowing and directly frozen in liquid nitrogen. Tissue was then pulverized in liquid nitrogen and transferred to TRIzol Reagent (Invitrogen, Carlsbad, CA) and RNA was isolated according to the manufacturer's instructions. RNA was then treated with RNase-free DNase (Qiagen, Valencia, CA) for 25 minutes at room temperature, ethanol precipitated and resuspended in nuclease-free water. Reverse transcription (RT) was performed using Advantage RT-for-PCR kit (Takara, San Jose, CA) following the manufacturer's instructions with 20 μM oligo (dT)₁₈. cDNA was diluted 1:10 in nuclease-free water for qRT-PCR.

qRT-PCR was performed using 2× PowerUp SYBR Green Master Mix (Applied Biosystems, Carlsbad, CA) as follows per well: 5 μL 2× PowerUp SYBR Green Master Mix, 0.75 μL cDNA (diluted 1:10), 1.25 μL nuclease-free water, and 3 μL combined 1.5 μM forward and reverse primers. All qRT-PCR reactions were performed in three technical replicates and all primers were tested for non-specific amplification using water and specificity using genomic DNA and analysis of melt curves. All reactions were run using the following program: 95° C. for 10 minutes; 40 cycles of 95° C. 30 sec, 55° C. 30 sec, 72° C. 30 sec. Melt curves were generated by heating the final PCR 1.6° C./s to 95° C. for 15 sec, decreasing the temperature to 60° C. at 1.6° C./s and slowly increasing back to 95° C. at 0.1° C./s. All primers are listed in TABLE 2.

TABLE 2
Oligonucleotide probes used for qRT-PCR and northern blot experiments.

| | SEQ. ID. | | |
|---|---|---|---|
| NAME | NO: | SEQUENCE | USE |
| Actin qRT-PCR Forward | 1 | CCTGAGGTCCTTTTCCAACCA | Amplify Actin for qRT-PCR |
| Actin qRT-PCR Reverse | 2 | GGATTCCGGCAGCTTCCATT | Amplify Actin for qRT-PCR |
| Nuclear PDS1 qRT-PCR | 3 | CATTCCGAGGCTTAATTTACCG | Amplify Nuclear PDS1 for qRT-PCR |
| Forward | | | |
| Nuclear PDS1 qRT-PCR | 4 | CTTTCAGTTCCCAACGAAGACC | Amplify Nuclear PDS1 for qRT-PCR |
| Reverse | | | |
| Nuclear PDS2 qRT-PCR | 5 | TTGGAATTGGTATTTGCACCTG | Amplify Nuclear PDS2 for qRT-PCR |
| Forward | | | |
| Nuclear PDS2 qRT-PCR | 6 | TTTTTGCTTTGCTCTGATCTGC | Amplify Nuclear PDS2 for qRT-PCR |
| Reverse | | | |
| PTS38, 39, 40 Sense | 7 | GGTGGAAAGGTAGCTGCATGGA | Detect sense transcription from |
| Northern blot probe | | | transgene on Northern blot |
| PTS38, 39, 40 Antiense | 8 | TCCATGCAGCTACCTTTCCACC | Detect antisense transcription |
| Northern blot probe | | | from transgene on Northern blot |

Results were first normalized to the housekeeping gene Actin (dC_{T}) and then normalized to wild-type (ddC_{T}). Relative quantity of ddC_{T} value was calculated as 2^{-(ddCt)}. Error bars represent standard error of the mean (SEM).

Experimental Results

Integration of Phytoene Desaturase cDNA Fragments into the Tobacco Chloroplast Genome

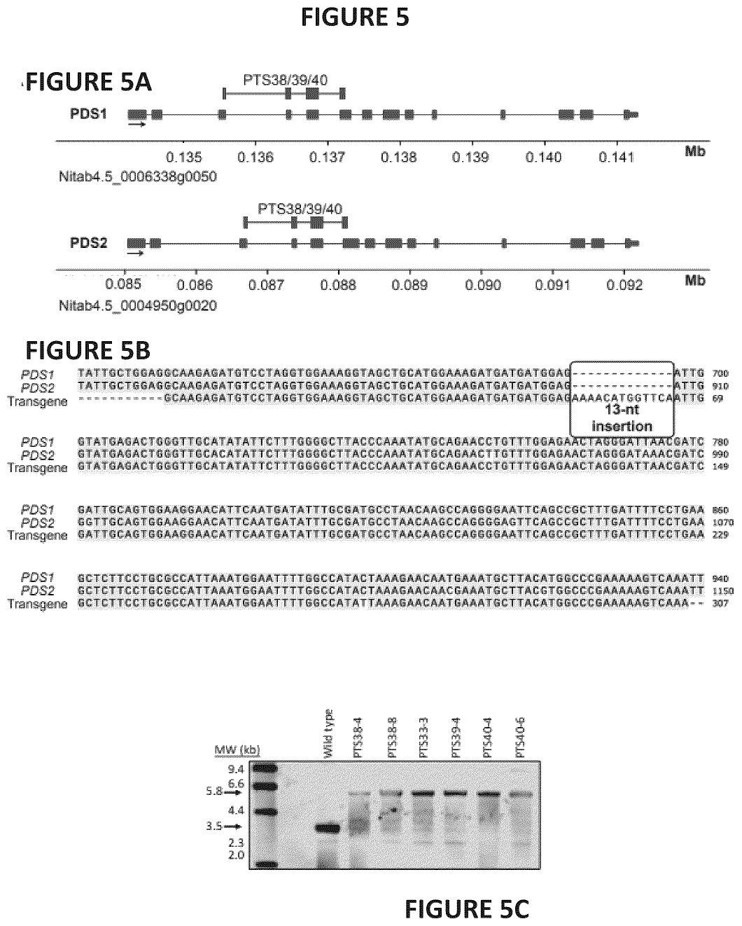

Based on homology search in GenBank, two apparent full-length cDNAs (XM_016610712.1 and XM_016642615.1) encoding Phytoene Desaturase (PDS) genes with >99% identity were identified in tobacco (Nicotiana tabacum). Subsequent search of SolGenomics database (www.solgenomics.net) using these two sequences confirmed N. tabacum genomic loci with 100% identity to the cDNA sequences (Nitab4.5_0006338g0050.1 and Nitab4.5_0004950g0020.1, termed below as PDS1 and PDS2, respectively). Two 300 nucleotide regions of the PDS1 cDNA (coordinates 645-938 and 1155-1434 based on the XM_016610712.1 transcript) were chosen for stable integration into the chloroplast genome. A lineup of the nucleotide sequences of the plastid transgenic sequences and their corresponding cDNAs are shown in FIG. 5A (for the PDS645-938 region).

The PTS40 chloroplast transformation vector expresses the PDS645-938 region from convergent chloroplast promoters, is shown to drive high-level accumulation of unprocessed dsRNA in chloroplasts (Zhang et al., 2015). As controls for possible effects of chloroplast dsRNA on nuclear PDS gene silencing, both sense- and antisense-oriented PDS645-938 region were expressed from a single chloroplast promoter, to create PTS38 and PTS39, respectively. As a further control, a second, more distal region of the PDS cDNA, PDS1155-1434, was also expressed as a dsRNA from convergent promoters in PTS41.

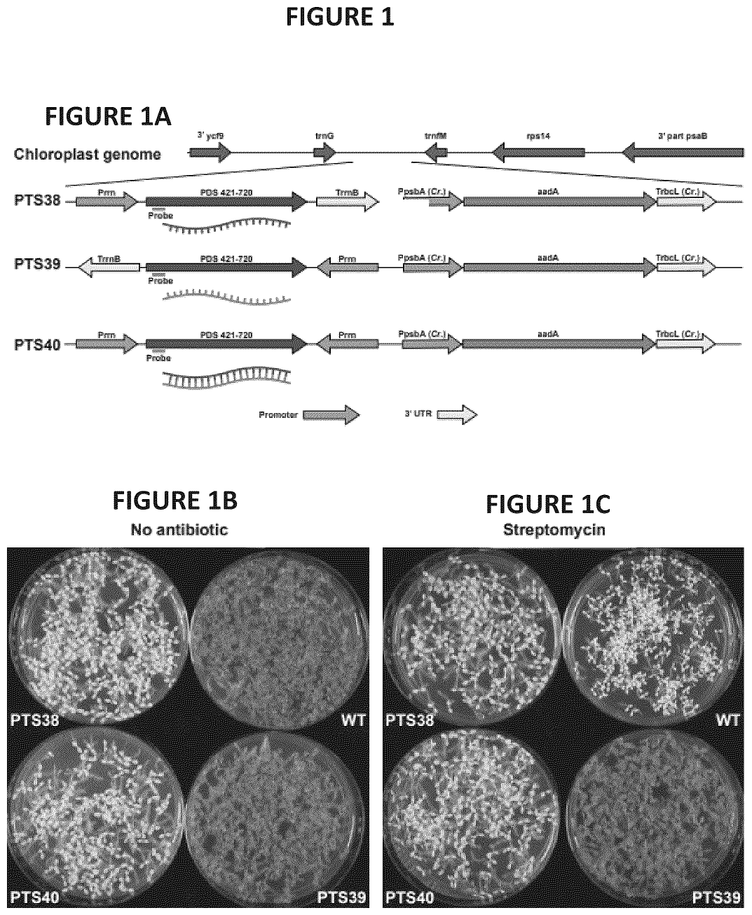

The PDS transgenes were cloned next to a selectable aadA spectinomycin resistance gene, between regions of identity to the tobacco chloroplast genome to mediate site-directed integration by homologous recombination (FIG. 1A). Tobacco chloroplast transformation via particle bombardment of in vitro-grown leaf tissue was performed according to standard procedures (Staub and Maliga, 1992) and transformants were selected on spectinomycin-containing medium. Leaf tissue from primary transformed shoots were used for two subsequent rounds of plant regeneration to ensure homoplasmy of the resultant transformed lines. Integration of the transgenes was confirmed in all transformed lines during the selection process by PCR and sequencing of amplicons.

Site-directed integration and homoplasmy of the transgenes was confirmed by Southern blot analysis, as confirmed in FIG. 5C using BglII digestion of total genomic DNA from wild-type and transplastomic seedlings. As can be seen in FIG. 5C, the insertion of the PDS and aadA transgenes into the plastid genome should result in a 5.7 kb band whereas wild-type plastids should reveal a 3.49 kb band. The hybridization pattern in FIG. 5C confirms homoplasmy of all transplastomic lines tested, as indicated by the sole presence of the 5.7 kb band in those lines and the bsence of the wild-type 3.49 kb band.

Phenotype of T0 Transplastomic Plants

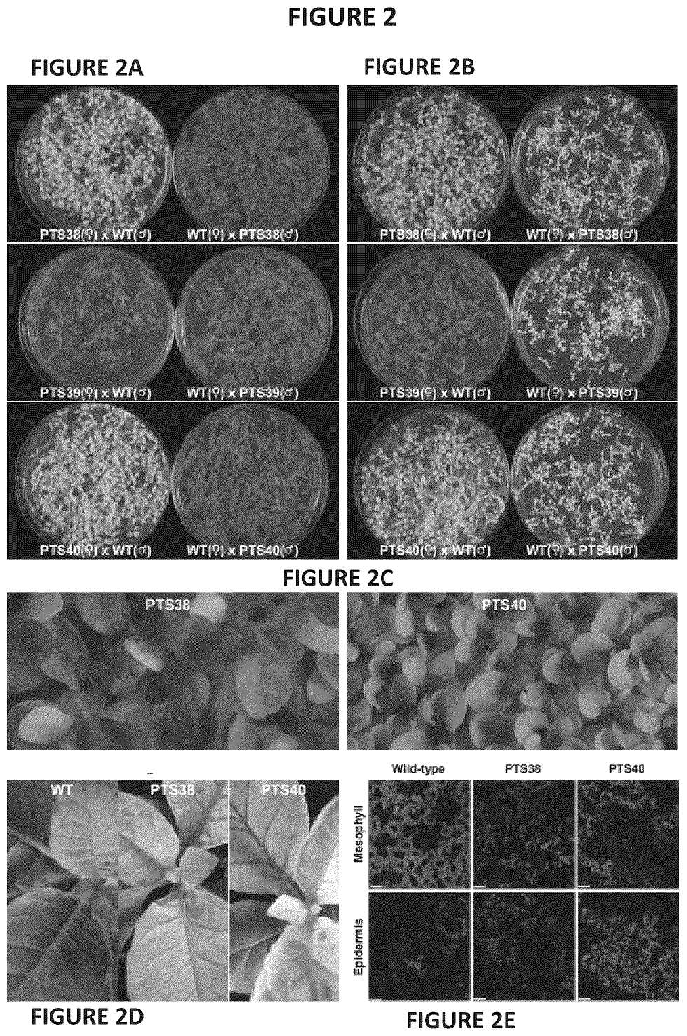

Chloroplast-transformed (transplastomic) lines grew normally in sterile tissue culture and had no apparent negative phenotype. T0 homoplasmic plants were rooted in tissue culture, then transferred to soil and placed in a growth chamber with cycling light to acclimate plants for subsequent greenhouse growth. After ˜10 days in soil, new leaves from PTS38 (sense) and PTS40 lines shown in FIG. 2D, and PTS41 dsRNA lines, but not PTS39 (antisense) lines, were surprisingly observed to be chlorophyll deficient. All independent transplastomic events for each of the PTS constructs that were transferred to soil had similar phenotypes, indicating the observed pigment deficiency was caused by the chloroplast transgenes. The chlorophyll deficient phenotype appeared uniform in new leaves, though with a yellow or very pale green appearance suggesting the presence of residual chlorophyll and/or other carotenoids. As the transplastomic plants continued to grow, subsequent new leaves were either uniformly bleached or appeared chimeric with irregular patterns of bleaching mixed with some nearly green sectors. The bleaching phenotype was most severe in PTS40 dsRNA lines, which grew most slowly and whose leaves were nearly completely bleached, with PTS38 phenotype being nearly as strong as PTS40 lines, while the PTS41 line was bleached to a lesser extent. In contrast, the PTS39 PDS antisense line was uniformly green and had no apparent phenotype, as in wild-type controls.

The pigment deficiency observed in the transplastomic plants suggested potential knockdown of the nuclear-encoded PDS gene function. However, knockdown of nuclear-encoded PDS genes via nuclear transformation technologies typically results in albino leaf tissues (Senthil-Kumar et al., 2007) due to lack of carotenoid accumulation and concomitant degradation of chlorophyll, suggesting some differences between plastid-expressed PDS gene silencing and the previously observed nuclear gene silencing approaches. Although the pigment deficient chloroplast-transformed lines grew much more slowly in the growth chamber than green plants, they were eventually transferred to the greenhouse to produce seed. Bleaching of leaves continued in the greenhouse and growth was slow, as expected. However, all plants eventually grew out of the bleaching phenotype, lateral branching occurred, and flowering was ultimately profuse with apparent normal seed set.

Maternal Inheritance of Plastid Transgenes and Chlorophyll Deficiency

To confirm the chlorophyll deficient phenotype was the result of the plastid-encoded PDS transgenes, maternal inheritance of the plastid-encoded traits was examined. Transplastomics lines were allowed to set self-seed in the greenhouse and were used for reciprocal crosses to wild-type plants. The progeny of selfed plants carrying the PDS645-938 region transgenes are shown in FIGS. 1B and C. As expected, wild-type seedlings are green on medium lacking antibiotics (as shown in FIG. 1B, WT on left) but are uniformly bleached white on medium containing spectinomycin (as shown in FIG. 1C; WT on right). Unexpectedly, seedlings of the PTS38 and PTS40 lines have an intermediate phenotype, being uniformly bleached yellow rather than white as in the antibiotic sensitive wild-type line. This result suggested the PTS38 and PTS40 lines are homoplasmic, but the pigment deficiency may be due to silencing of the nuclear-encoded PDS genes rather than antibiotic sensitivity. Seedlings of the PTS39 antisense PDS line were uniformly green, indicating uniform resistance to the antibiotic as expected for a homoplasmic chloroplast-encoded aadA transgene, but no pigment deficiency was observed in these lines. PTS41 seedlings carrying the PDS1155-1434 region were uniformly resistant to the antibiotic, and were only slightly delayed in greening (data not shown, see below) compared to wild-type seedlings in the absence of antibiotics.

To confirm the pigment deficient phenotype of PTS38 and PTS40 lines is due to PDS gene silencing, seeds from selfed plants were also sown in the absence of antibiotics (FIG. 1B). As expected, wild-type seedlings are green. In contrast, PTS38 and PTS40 seedlings still had a uniform pigment deficient phenotype, again indicating that these lines are homoplasmic for the chloroplast traits and confirming the bleaching phenotype is chloroplast-encoded, apparently due to silencing of the nuclear-encoded PDS gene. The PTS39 lines were uniformly green, indicating no apparent effect of this chloroplast-encoded antisense PDS transgene on nuclear PDS gene silencing.

Reciprocal crosses of chloroplast-transformed lines with wild-type plants is typically used to prove maternal inheritance of a chloroplast transgenic trait. As shown in FIGS. 2A, when the PTS38 and PTS40 plants were used as female parents in a cross to wild-type plants, all seedlings are uniformly pigment deficient on media lacking antibiotics. In contrast, when the plastid transformed lines are used as the male pollen donor in crosses to wild-type plants, all seedlings are uniformly green. These results confirm that the chlorophyll deficient phenotype is maternally inherited and not transmitted through pollen, as expected for a chloroplast-encoded trait. Maternal inheritance was further confirmed in T2 and T3 generations of the PTS40 and PTS38 lines (data not shown), confirming that there is no active copy of the chloroplast PDS transgenes in the nuclear genome.

Although PTS38 and PTS40 T1 seedling cotyledons were uniformly chlorophyll deficient, the first true leaves emerged as partly green as shown in FIGS. 2C, and subsequent leaves were uniformly green in tissue culture. The presence or absence of sucrose in the medium did not affect this phenotypic outcome (data not shown), indicating that the pigment deficiency is apparently subject to developmental timing. However, when PTS38 and PTS40 T1 plants that were grown in tissue culture to the 4-5 leaf stage were then transferred to soil, the chlorophyll deficient phenotype of newly emerged leaves quickly returned (as shown in FIG. 2D). Interestingly, PTS41 T1 plants derived from seedlings that were green in tissue culture medium also had newly emerged leaves that became chlorophyll deficient when placed in soil, albeit with a much less intense and transient occurrence compared to PTS38 and PTS40 lines. These latter observations suggest that the PDS1155-1434 dsRNA transgene in PTS41 lines is less effective at PDS knockdown than in plants expressing the PDS645-938 dsRNA (PTS40) or sense RNA (PTS38) transgenes.

Chloroplast Division is Blocked in a Tissue-Specific Manner in Chlorophyll Deficient Lines

To further understand the pigment deficient phenotype of transplastomic plants, plastid morphology in leaves from T1 plants grown in soil was examined by confocal microscopy. As can be seen in FIG. 2E, mesophyll cells of wild-type plants (and PTS39 lines, data not shown) have characteristic large chloroplasts (average size ˜5 uM) arranged along the periphery of each cell. Chloroplasts appear fully developed and densely packed side-by-side. In contrast, PTS38 and PTS40 transplastomic lines contain smaller (average size ˜2.5 uM) plastids, more loosely arranged mostly along the periphery of the cell. Interestingly, most of the plastids appear as closely associated pairs, suggesting a block in plastid development shortly after plastid division.

In contrast to chloroplasts of mesophyll cells, epidermal cell plastids were observed to be relatively small (˜3 uM average size) and loosely arranged along the cell periphery in wild-type, PTS38 and PTS40 lines. These results suggest that PDS gene knockdown may not occur in epidermal cells of the plastid transformed lines. We speculate that lack of PDS gene knockdown in epidermal cells may allow some chlorophyll to accumulate in those cells, resulting in the yellow appearance of PTS38 and PTS40 lines rather than albino bleaching phenotype typically observed in nuclear-encoded PDS knockdown lines.

Q-RT-PCR Confirms Knockdown of PDS mRNA in Plastid Transformed Lines

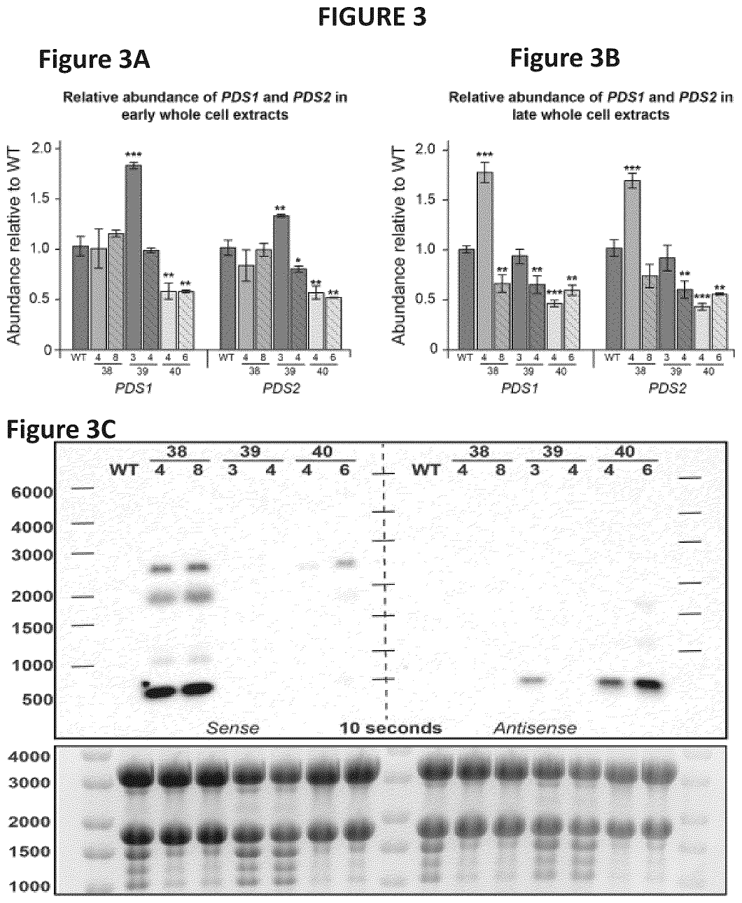

Pigment deficiency of chloroplast transformed lines carrying PDS dsRNA or sense-strand transcripts suggests that chloroplast-transgene-encoded RNAs exert gene silencing effects on nuclear-encoded cytoplasmically-localized PDS gene mRNAs. To directly confirm knockdown of the nuclear-encoded PDS genes, quantitative reverse-transcriptase PCR (Q-RT-PCR)-mediated analysis of total cellular RNA from T1 transplastomic and wild-type 12-day old seedlings was performed. PCR primer pairs that map outside of the chloroplast transgenes were used to distinguish only the nuclear-encoded PDS genes. As shown in FIG. 3A, in 12-day-old seedlings, when cotyledons of PTS40 and PTS38 lines are uniformly bleached, the level of PDS1 mRNA is significantly reduced to about half and PDS2 mRNA is reduced slightly in the PTS40 dsRNA lines compared wild-type. In contrast, PDS1 and PDS2 mRNA appears similar to wild-type green seedlings in the other plastid-transformed lines that do not display the bleaching phenotype. As shown in FIG. 3B, at the later development (24-day-old seedlings) stage when the first true leaves of PTS40 seedlings are green, nuclear-encoded PDS1 mRNA levels recover to near wild-type levels, consistent with the cessation of the pigment-deficient phenotype. These results confirm that the pigment-deficient phenotype of PTS40 lines is due to knockdown of the nuclear-encoded PDS mRNA, while the bleaching in the PTS38 sense lines is likely due to a different post-transcriptional gene silencing mechanism.

Northern Blot Analysis Confirms High-Level Expression of Plastid-Encoded PDS Transcripts

To confirm that plastid-encoded PDS transgenes accumulate PDS transcripts and determine their strand orientation, Northern blot analysis of total cellular RNA was performed, as shown in FIG. 3C, using strand-specific oligonucleotide probes. As shown in the top of FIG. 3C, the PDS transgenes are flanked by the aadA transgene downstream and plastid-encoded genes upstream of the PDS transgenes. Transcription termination is known to be inefficient in plastids (Bock, 2013), thus readthrough polycistronic transcripts are predicted.

Using a sense-PDS-strand probe, abundant transcripts are observed in both PTS38 and PTS40 lines, but not in PTS39 lines. The expected ˜300 nucleotide sense-strand transcript derived from the PDS transgene fragment accumulates to high levels in the PTS38 lines and to a lesser extent in the PTS40 lines. Higher molecular weight transcripts are observed in all lines, indicating the presence of readthrough transcripts, as predicted. Using the antisense probe, both PTS39 and PTS40 lines accumulate the ˜300 nucleotide antisense PDS transcript. Together, these results indicate that PTS40 lines accumulate both sense and antisense PDS transgene-derived transcripts, suggesting that dsRNA accumulates in those lines. In contrast, and as expected, the PTS38 lies accumulate only sense PDS transgene-derived transcripts and the PTS39 lines accumulate only antisense-PDS transgene-derived transcripts. It should be noted that transcripts for the endogenous nuclear-encoded PDS1 and PDS2 genes are expected to be expressed at much lower levels than the plastid transgenes and are not observed on the northern blot.

Chloroplast Transgene-Encoded Small RNAs Accumulate to High Levels

Silencing of the nuclear-encoded PDS mRNA via plastid-encoded transgenic RNAs suggests the latter can enter the host gene silencing pathway, characterized by the presence of 21-24 nt small RNAs. Since previous reports of chloroplast-transformed plants carrying dsRNA transgenes did not observe any apparent small RNA accumulation by northern blot analysis (Zhang et al. 2015; Bally et al., 2016), we sought to characterize the small RNA population in the transplastomic plants via small RNA sequencing. Total cellular RNA was extracted from 12-day old bleached (PTS38 and PTS40) and green (PTS39) T1 seedlings and wild-type controls derived from self-pollination, small RNA libraries were prepared and sequenced via the Illumina high throughput sequencing platform.

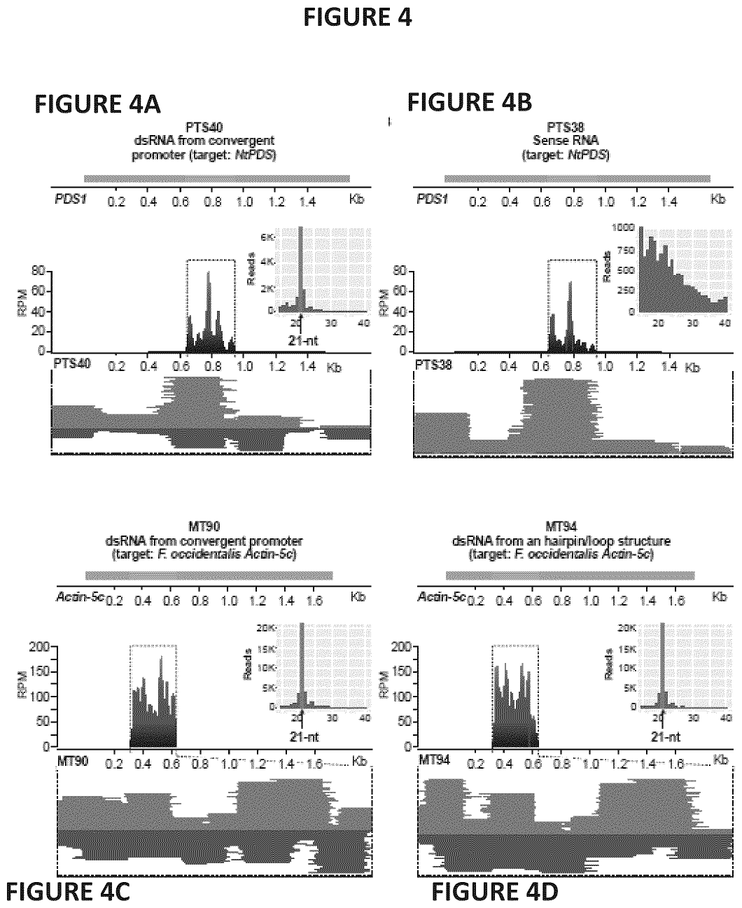

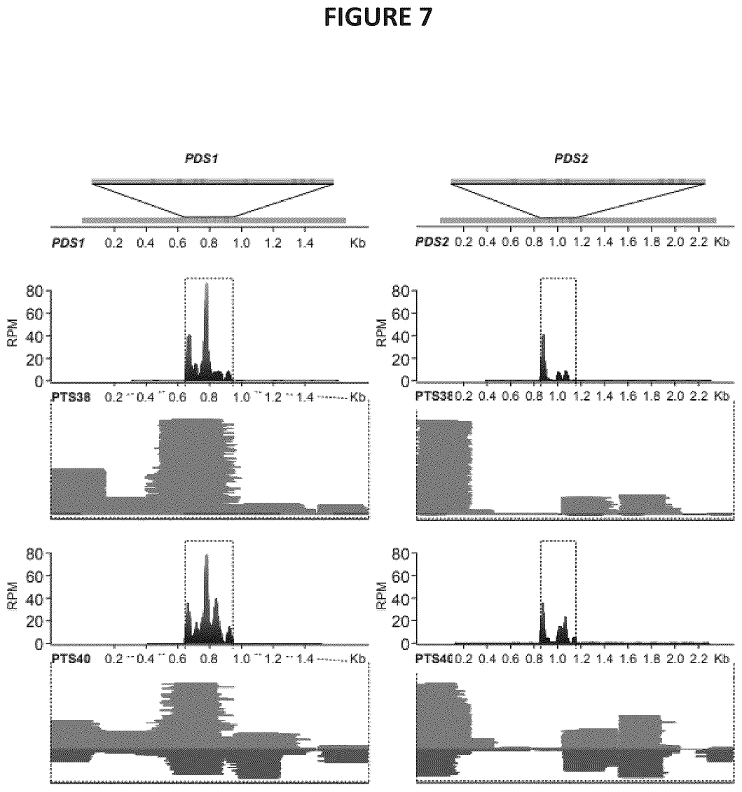

As expected, no small RNAs mapping to either PDS1 or PDS2 nuclear genes were detected in wild-type (green) seedlings. In contrast, FIG. 4 shows that small RNAs that map to PDS nuclear genes were observed in transplastomic lines derived from multiple PTS transgenes, albeit with very different accumulation patterns. As anticipated from their bleached phenotype, both PTS38 and PTS40 lines accumulate large numbers of small RNAs (up to 7000 reads) that map to both PDS nuclear genes. In contrast, only one (green) PTS39 line (PTS39-3) accumulated any detectable small RNA reads, suggesting an amount of small RNA accumulation in these lines that is below the threshold required to catalyze a gene silencing response under the conditions tested. In contrast, only one (green) PTS39 line (PTS39-3) accumulated any detectable small RNA reads, suggesting an amount of small RNA accumulation in these lines that is below the threshold required to catalyze a gene silencing response under the conditions tested. Inspection of the siRNA sequences in FIG. 7 indicates that none mapped to polymorphic PDS2 sequences, and no secondary siRNAs derived from the nuclear-encoded PDS genes beyond the 294 nt plastid PDS transgene region were observed, suggesting that the siRNAs derive directly from plastid transcripts. The small RNA abundance and accumulation pattern was consistent across independent transplastomic events for PTS38 and PTS40 lines, indicating a reproducible response to the plastid-derived gene silencing effect.

21 nt Phased Small RNAs Accumulate from the Chloroplast PDS dsRNA Construct

The abundant small RNAs that map to the plastid-expressed PDS transgenes was unexpected, according to all previous reports (Zhang et al., 2015; Bally et al., 2016; Zhang et al., 2017; Bally et al., 2018). To provide insight to the mechanism of chloroplast-transgene-derived small RNA biogenesis, their size distribution, strandedness and potential phasing was examined, as shown for representative lines in FIGS. 4A and 4B. For the PTS40 dsRNA transplastomic lines, small RNAs derived from total cellular RNA of 12-day old T1 seedlings map abundantly to both strands of both nuclear PDS genes (FIG. 4A). Although the size distribution of small RNAs in these lines extends from 15 nt to longer than 30 nt length, 21 nt-sized small RNAs make up the vast majority of the reads across both DNA strands. Furthermore, the small RNA mapping pattern shows periodicity on both DNA strands resulting in a significant phasing score of >35 for reads that map to the PDS1 gene. Together, these results indicate that chloroplast-expressed mRNAs can enter the gene silencing pathway in the cytoplasm, and that 21 nt phasiRNA is the predominate form of small RNAs derived from the PTS40 transgene dsRNA.

Abundant small RNAs were also mapped in the transplastomic PTS38 sense strand lines to both PDS genes, as shown in FIG. 4B. However, in contrast to the PTS40 dsRNA transplastomic lines, PTS38-derived small RNAs map exclusively to the sense strand of the PDS genes. Similar to PTS40 lines, no small RNA reads mapped outside of the PDS gene region corresponding to the transgene. Further, no significant phasing score was observed in these lines and the size distribution of small RNAs was broad from 15 to >30 nt length, with similar abundant read counts in the size range from ˜15 to 24 nts. Consistent read numbers and distribution patterns across independent events confirmed these results and suggest a different mechanism of nuclear PDS gene silencing from the PTS38-derived sense-strand compared to the PTS40 dsRNA lines.

21 Nt phasiRNAs Accumulate in the Cytoplasm

Accumulation of 21 nt phasiRNA in the PTS40 lines must result from processing of plastid-derived transcripts in the cytoplasm after movement by unknown mechanisms. However, to determine if plastids also accumulate transgene-derived 21 nucleotide phased small RNAs and to rule out previously uncharacterized RNA processing events of transgenic transcripts inside the plastid, we prepared small RNA libraries from purified plastids from the same 12-day old transplastomic T1 seedlings as described above. Plastids were purified using a commercially available size exclusion column from bleached seedlings of the PTS40 and PTS38 lines as well as wild-type (green) seedling controls. Using this approach, contaminating mitochondrial RNAs were observed to be less than 5% of total reads, nuclear-encoded miRNAs were depleted on average by >95% and chloroplast transcripts were enriched ˜100-fold as compared to whole cell RNA extractions as shown in Table 1, indicating a highly enriched chloroplast fraction was obtained.

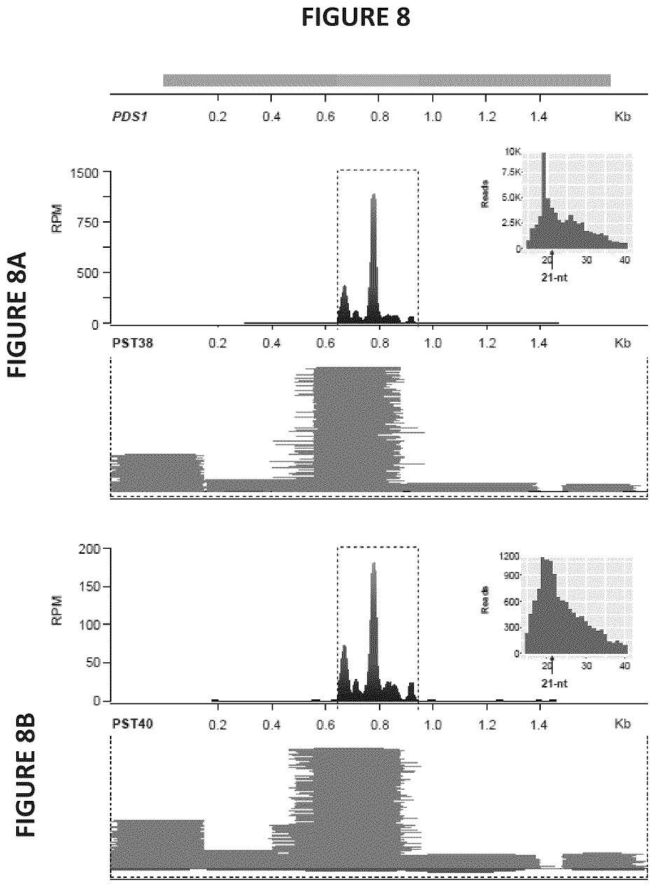

FIG. 8 shows the small RNA read mapping results observed from purified plastids. PDS transgene-derived small RNAs accumulate in purified plastids from transplastomic PTS40 lines but with a different pattern from that seen when using total cellular RNA. Although the plastid-localized small RNAs are abundant and distribute across the PDS region covered by the transgene, the small RNAs map almost exclusively to the sense-strand of the transgene-encoded PDS1 gene and few small RNA reads map to the antisense PDS strand. Furthermore, a broad size distribution of the small RNAs exists and there is no evidence for phasing of the small RNA population or addition of nucleotides to the 3′-end of transgene border small RNA reads. These results confirm that the 21 nucleotide phasiRNAs arise in the cytoplasm and therefore derive from plastid transcripts that move to the cytoplasm. Inspection of the reads in both the chloroplast and total RNA fraction indicates significant overlap, as expected (data not shown).

The mapping of small RNA reads from the purified chloroplast fraction in the PTS38 transplastomic seedlings more closely resembles its accumulation pattern observed from total cellular RNA. All chloroplast small RNAs map exclusively to the sense strand of the PDS1 gene, as expected. No phasiRNAs or nucleotide additions were observed. Again, inspection of the reads in both the chloroplast and total RNA fraction indicates significant overlap, as expected (data not shown).

Processing of Chloroplast-Transgene-Derived dsRNA to 21 nt phasiRNAs is a Universal Phenomenon

The finding of abundant 21 nt phasiRNAs derived from PDS dsRNA raised the question of whether a nuclear-encoded homology target is required for processing. Furthermore, widespread processing of nonhomologous plastid-expressed dsRNA to 21 nt small RNAs in the cytoplasm would significantly broaden the applications of plastid transgenesis to new pathogenic species and targets. To examine these questions, we utilized transplastomic tobacco plants that express dsRNA against non-host (insect) targets, from either convergent chloroplast Prrn promoters identical to the PDS dsRNA transgene construct (MT90 and MT91) or via a single chloroplast Prrn promoter driving a more traditional hairpin stem/loop dsRNA construct (MT94 and MT95) that carries 2 copies of the target gene fragment separated by an intron that is not processed in chloroplasts.

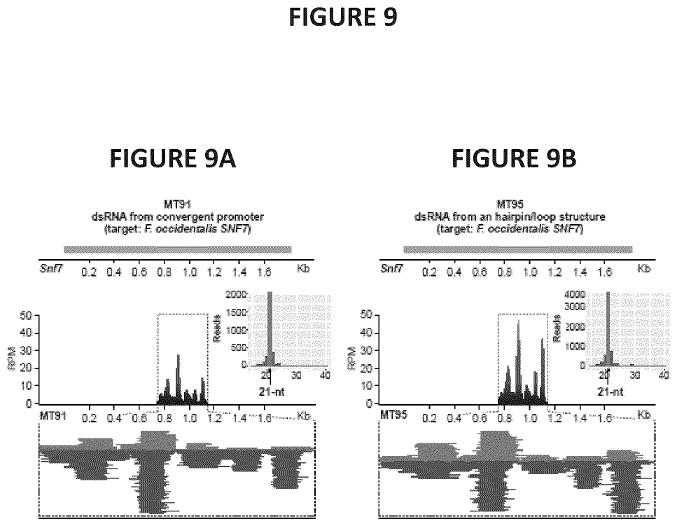

FIGS. 4C and 4D and FIG. 9 shows examples of the mapping of small RNAs across insect (F. occidentalis) gene targets, SNF7 and Actin5C, from total cellular RNA extracts from 12-day old seedlings of representative transplastomic plant lines expressing dsRNA against these gene targets. The results of the small RNA mapping are similar from both convergent chloroplast promoters and the hairpin stem/loop dsRNA constructs, and across 2 independent transplastomic lines tested for each construct. In all cases, small RNAs are abundant and map across both DNA strands of SNF7 and Actin5C, along the entire gene region covered by the plastid transgenes, with no apparent transitive small RNAs mapping outside of this region. Importantly, the size distribution of the small RNAs show a strong peak at 21 nt in all events, and a significant phasing score of −27-62 in nearly all constructs and events is observed, though the MT91 events have a slightly weaker phasing score. These results confirm that plastid-expressed dsRNA enters the host cytoplasmic RNAi pathway to generate 21 nt phasiRNAs independent of host homologous sequences and independent of the transgene method used to make plastid-expressed dsRNA.

Plastid-Encoded Transgene Polycistronic Transcripts are Processed into Small RNAs

The Northern blot analysis shown in FIG. 3C indicates that transcripts from the plastid-encoded PDS dsRNA and sense-strand transgenes do not completely terminate at the end of the plastid transgenes and transcription can readthrough to the downstream aadA transgene, creating polycistronic PDS-aadA transcripts. Transcript termination is known to be inefficient in plastids, so this result is not surprising. Surprisingly, though, small RNAs that map to the aadA and other downstream transgenic sequences also accumulate in both the PTS38 and PTS40 lines, as shown in FIG. 10. These results indicate that it may be possible to use multiple transgenic target sequences to silence multiple nuclear genes using a single plastid-encoded transgenic sequence, as small RNA production occurs throughout the polycistronic transcript.

Surprisingly, small RNAs are produced from both strands of the downstream aadA transgene, even though a dsRNA construct is not used to drive expression of the aadA gene. This result further indicates that plastid-encoded transcripts must be transported to the cytoplasm, as small RNAs derived from the antisense strand of aadA can only arise from entry into the cytoplasmic gene silencing pathway.

The ability of chloroplast-encoded transgenes to enter the host gene silencing pathway and knockdown the expression of a nuclear-encoded gene is a surprising and novel finding. To our knowledge, this is the first report of a chloroplast-transgene-derived transcript that exerts its function in a different cellular compartment, apparently via movement out of the organelle to the cytoplasm. Our findings raise the question of whether other plastid-transgene-encoded transcripts or proteins may be engineered to exert additional functions outside of the organelle and opens the field of chloroplast engineering to a large number of new trait targets encoded in the nuclear genome and functioning in different cellular compartments.

The processing of plastid-transgene-encoded dsRNAs to 21 nt phasiRNAs appears to be universal and does not require a plant host-encoded transcript target to enter the RNAi pathway, as evidenced by similar small RNA profiling from the PDS nuclear gene or non-host insect control dsRNA plastid transgenes. Furthermore, movement of the plastid transcripts to the cytoplasm is a prerequisite for 21 nt phasiRNA production, since the gene silencing machinery does not exist in plastids. Taken together, our results indicate that movement of transcripts out of the plastid compartment must happen commonly.

The movement of plastid-expressed RNAs out of the organelle may be developmentally regulated as the initial triggering events originate in germinating seedling cotyledons and plants that are newly transferred from sucrose-containing medium to soil. The first true seedling leaves emerge partly green and subsequent plant growth in tissue culture looks normal. In contrast, the bleaching of new leaves of transplanted plants transferred to soil is persistent for several weeks, suggesting a continual turnover of a subpopulation of plastids during leaf development. This phenotype is reminiscent of autophagic turnover of plastids, perhaps initially in response to limited photosynthetic activity that induces sugar starvation or via the RCB pathway that affects chloroplast stroma (reviewed in reviewed in (Izumi et al., 2019; Woodson, 2019; Izumi and Nakamura, 2018; Zhuang and Jiang, 2019). We speculate that accumulating redox or other plastid signals during these developmental stages may trigger autophagy, and plastid contents become available to the cytoplasm en route to the vacuole by unknown means. During this transport process, highly expressed plastid transgene RNAs are apparently stable enough to be captured by the gene silencing apparatus located in the cytoplasm. It is also possible that the small RNAs produced from the plastid transgenes may be mobile between or outside of cells, thus spreading the PDS gene knockout phenotype. However, an attempt to transfer the bleaching phenotype from a PTS40 transplastomic event to wild-type tobacco plants via grafting was unsuccessful.

Abundant small RNAs derived from plastid transgenes also accumulated in purified chloroplasts. Small RNA profiling data according to the present disclosure also revealed abundant small RNAs for the antibiotic selectable marker, aadA, used for plastid transformation and several other endogenous chloroplast genome sequences. The small RNA profiles were not highly consistent across independent events, and the small RNA sizes were broadly distributed and not phased, suggesting these sequences are likely intermediates in the process of transcript degradation. Several reports have identified small- and long-non-coding RNA in chloroplasts, however, no direct role for these in chloroplast gene regulation has been clearly shown and no role of chloroplast small RNAs in transcriptional gene silencing has been reported (reviewed in Anand and Pandi, 2021). Interestingly, there is a recent report of plastid tRNA-derived non-coding RNA fragments located in the cytoplasm but not in chloroplasts, though no function of these was identified (Cognat et al., 2017). It is assumed the plastid transgene-derived small RNAs observed in purified chloroplasts do not contribute to the production of 21 nt phasiRNAs in the cytoplasm, which, without being bound by theory, may arise from longer or full-length transcripts liberated to the cytoplasm. Additional analysis will be needed to determine if plastid-localized small RNAs processed from endogenous plastid genes may play a role outside of the organelle, perhaps as part of the chloroplast retrograde signaling pathway (reviewed in Jan et al., 2022; Liebers et al., 2022).

Liberation of plastid-transgene-derived transcripts to the cytoplasm occurred in the absence of any specific treatments designed to facilitate the process, indicating that movement of plastid transcripts to the cytoplasm occurs naturally. The instant observations are different from previous reports of “escape” of DNA from mitochondria or chloroplasts (Thorsness and Weber, 1996; Huang et al. 2003; Bock and Timmis, 2008; Bock 2017) where a strong antibiotic selection is required to identify rare transfer of a chloroplast transgene to the nucleus. Likewise, the instant disclosure is in contrast to a report that utilized paraquat herbicide or bacterial infection to catalyze reactive oxygen species that disrupt chloroplast membranes, thus allowing leakage of a chloroplast-localized fluorescent protein to the cytoplasm (Kwon et al., 2013). Regardless of the mechanism that moves chloroplast-transgene-encoded transcripts to the cytoplasm, the instant results show that these transcripts are processed to 21 nt phasiRNAs and enter the host RNAi pathway. Plastid-encoded PDS RNAs affecting nuclear PDS gene silencing via multiple lines of evidence were observed, including accumulation of 21-nt phasiRNAs that derive from Dicer-like processing of dsRNA in the PTS40 and MT lines (FIGS. 4 and 9). Interestingly, lack of siRNAs spreading to adjacent PDS1 sequences or siRNAs derived from the PDS2 polymorphic regions suggests that plastid-encoded transcripts can be used directly as template for Dicer cleavage without RDR activity. In support of this conclusion, the processing of plastid transgene-encoded dsRNAs to 21-nt phasiRNAs does not require a plant host-encoded transcript target to enter the RNAi pathway, as evidenced by abundant phasiRNAs derived from the dsRNA plastid transgenes encoding an insect gene fragment with no cognate nuclear-encoded complimentary partner. The data thus suggest a departure from the current understanding of plant phasiRNA biogenesis, which typically initiates with an Argonaute-catalyzed (AGO) cleavage of a single-stranded mRNA precursor, then converted to dsRNA by an RDR protein, followed by processing into 21-nt or 24-nt RNA duplexes by a Dicer-like (DCL) protein (Hung and Slotkin, 2021; Liu et al., 2020; Fei et al., 2013). On the other hand, also observed was apparent translational inhibition from sense-strand RNAs in the PTS38 line, as evidenced by pigment deficient plants with no PDS RNA knockdown via qRT-PCR (FIG. 3) or phasiRNAs by small RNA sequencing (FIG. 4). Translational repression of nuclear genes may occur by interfering with translationally active ribosomes via multiple mechanisms in response to accumulation of translatable transcripts at high concentrations (Que et al., 1997) as is observed in the PTS38 line by northern blot (FIG. 2).

While the precise molecular mechanisms leading to nuclear gene silencing from plastid transgenes will require additional research to fully understand, these results have dramatic implications for expanding the range of commercial targets available for plastid engineering. To date, only plastid-expressed long dsRNA has been used for TK-RNAi with insecticidal activity against a small number of Coleopteran and Lepidoptera insect pests. The ability to make abundant plastid transgene-derived small RNAs open new opportunities to target additional plant pests that are susceptible to small RNAs (reviewed in Hudzik et al., 2020; Koch and Wassenegger, 2021; Zhao et al., 2021), including fungi (Niu et al., 2021; Qiao et al., 2021), aphids Sattar and Thompson, 2016; Biedenkopf et al., 2020), plant viruses (Gaffar and Koch 2019) and nematodes (Medina et al., 2017; Tian et al., 2019). Furthermore, the ability to silence plant nuclear genes from the chloroplast now enables crop improvement via engineering a vast array of host metabolic and other processes (reviewed in Mansoor et al., 2006; Saurabh et al., 2014), including biotic and abiotic stress (Rajput et al., 2021), biomass (Feldmann, 2006) and grain yield (Feldmann, 2006; Shomura et al., 2008) with the advantages that plastid transformation brings of gene containment, lack of gene silencing and easy breeding via maternal inheritance.

### Example: Beneficial Nuclear Traits Generated Via Gene Silencing

Gene silencing via dsRNA has been used to create beneficial and agronomically important traits. Examples, as shown in Table 3 below, include knock out of knock down of genes involved in modifying plant metabolic pathways to enhance nutrient content and reduce toxin production, create agronomic traits of importance to farmer and consumers, and traits involved in breeding and plant production. In each case, the desired nuclear gene may be silenced via expression of a double-strand or sense-strand RNA plastid-encoded transgene with homology to the nuclear-encoded gene. As observed above, such plastid-encoded transcripts are predicted to leave the chloroplast and produce small RNAs in the cytoplasm, that will catalyze silencing of the cognate nuclear gene. As stated above, multiple nuclear-encoded genes may be silenced at the same time, by using a plastid transgene that encodes the target sequence for multiple nuclear genes.

TABLE 3

| | | | Example |
|---|---|---|---|
| Benefit | Trait | Gene | plant host |
| Enhanced | β-Carotene and | NCED1 | Tomato |
| nutrients | lycopene | | |
| | Carotenoid | ε-CYC | Brassica |
| | | | napus |
| | Carotenoid and | DET 1 | Tomato, |
| | flavonoid | | Brassica |
| | β-Carotene and | BCH | Potato |
| | lutein | | |
| | Starch | AtGWD | Maize |
| | Starch | AtGWD and | Arabidopsis |
| | | AtSEX4 | |
| | Oil quality | Fad2 | canola, peanut, |
| | | | cotton |
| | Increased | ZLKR and SDH | Maize |
| | Lysine | | |
| | Increased | Maize zein | Maize |
| | Lysine | storage protein | |
| | Lycopene | Lyc | Tomato |
| | Amylose | SBE IIa and | Wheat |
| | | SBE IIb | |
| | Amylose | SBE IIa and | Barley |
| | | SBE IIb | |
| | Stearic- and oleic- | Stearoyl-acyl- | Cotton |
| | fatty acids | carrier protein | |
| | | SAD1 | |
| Reduced | Reduced nicotine | CYP82E4 | Tobacco |
| toxins | Caffeine | CaMXMT 1 | Coffea |
| | | | canephora |
| | Cadmium | PCS | Rice |
| | Morphine | Codeine | Opium poppy |
| | | Reductase (COR) | |
| | Reduced arsenic | ACR2 | Arabidopsis |
| | Reduced | Arah2 | Peanut |
| | allergenicity | | |
| | Reduced | Lolp1, Lolp2 | Ryegrass |
| | allergenicity | | |
| Agronomic | Early ripening | LeETR4 | Tomato |
| traits | Delayed ripening | ACC oxidase | Tomato |
| | Flower colour: | CHS | Torenia |
| | blue to white | | hybrida |
| | Scent profile | PhBSMT | Petunia |
| | modification | | |
| Breeding traits | Parthenocarpy | AUCSI A | Tomato |
| | Parthenocarpy | CHS | Tomato |
| | Male sterility | TA29 | Tobacco |
| | Male sterility | GEN-L | Rice |
| | Male sterility | BCP1 | Arabidopsis |
| | | | thaliana |
| | Fertility restored | orfH522 | Tobacco |
| | | | (male sterile) |

### Example: Plastid-expressed pathogen gene silencing

In addition to beneficial traits created via silencing of plant host encoded genes, plastid-encoded dsRNA has been used to silence essential genes in plant pathogens and provide resistance of plants to those pests. Recent studies indicate that transplastomic plants can accumulate high levels of long unprocessed dsRNA that are effective against some Coleopteran and Lepidopteran insect pests (Zhang et al., 2015; Jin et al., 2015; Bally et al., 2016). In each of these reports, no apparent small RNA production from chloroplast transgenes was observed via Northern blots, leading the authors to conclude that the RNAi machinery is absent from plastids. Data indicates that processing of long dsRNA to siRNA does occur and therefore may not be a limitation for controlling plant pathogens that preferentially take up siRNAs, for example, viral, nematode and some fungal and oomycete pathogens (Liu et al., 2019; Jaubert-Possamai et al., 2019; Qiao et al., 2021; Taliansky et al., 2021).

For management of fungal diseases, RNAi-based gene silencing strategy offers enormous potential as an alternative to conventional fungicides. Several studies have provided evidence that cross-kingdom RNAi can occur between plant hosts and their fungal pathogens (4,5). Plant nuclear transgene-derived double-stranded RNA (dsRNA) can induce gene silencing in multiple fungal pathogens leading to decreased infections rates and disease symptoms. This approach has been successfully exploited for control of diverse fungal pathogens (reviewed in Wang, M., and Dean, R. A. 2019) including Botrytis cinerea (Wang et al, 2016), Fusarium graminearum (Koch et al. 2013; Hofle et al. 2019) and Fusarium oxysporum (Bharti et al. 2017), and Sclerotinia sclerotiorum (Andrade et al. 2016). Fungal gene silencing can be induced by long dsRNA or small RNAs applied directly to the fungus in vitro or via spraying of inoculated plants (spray-induced gene silencing, SIGS). In the case of Botrytis cinerea, application of either long dsRNA or small RNAs to Arabidopsis or tomato plants showed efficacy against gray mold disease (Koch et al. 2016b, McLoughlin et al. 2018). These results indicate that the fungus can efficiently take up either long dsRNA or processed small RNAs as an effective initiator of the RNAi process.

Genes encoding core components of the fungal RNAi machinery, Dicer (DCL) and Argonaut (AGO, AGL), have themselves been effective targets for gene silencing, leading to significant reductions in fungal growth, colonization, and pathogenesis. Although there is some functional redundancy in each member of these gene families, knockdown via SIGS (Werner et al. 2019) or knockout via mutagenesis of the fungus (Gaffar et al 2019) confirm that AGO2 and DCL1 genes of Fusarium graminearum are required for full pathogenesis. In contrast, knockout mutation of AGL2 (AGO1 homolog) but not AGL4 (AGO2 homolog) of Sclerotinia sclerotiorum significantly reduced pathogenesis on canola leaves (Neupane et al. 2019) while disruption of both DCL1 and DCL2 genes was required to prevent colony formation on leaves of canola, soybean or sunflower (Mochama et al. 2018).

Genes for Sclerotinia sclerotiorum AGL2 (Ss1G_00334), AGL4 (Ss1G_11723), DCL1 (Ss1G_13747) and DCL2 (Ss1G_10369) have been described (Neupane et al. 2019; Mochama et al. 2018). The Fusarium graminearum AGO1 (FGSG_08752), AGO2 (FGSG_00348), DCL1 (FGSG_09025) and DCL2 (FGSG_04408) cDNA sequences were used to blast the full genome sequence of Fusarium virguliforme Mont-1 in Genbank (AEYB00000000.1). Tblastx application was used to translate the Fusarium graminearum sequences in all reading frames against the Fusarium virguliforme Genbank sequence similarly translated in all reading frames. For each cDNA query, a single genomic contig in Fusarium virguliforme was identified with strong identity. The Fusarium virguliforme genomic DNA contig was further analyzed using the GENSCAN program (http://argonaute.mit.edu/GENSCAN.html) to parse out a predicted cDNA sequence.

Based on potential overlapping functions of AGO and DCL genes we decided to target both AGO/AGL and both DCL genes in single dsRNA constructs. Sequences of about 300 nucleotides from each gene were chosen for creating plastid-expressed RNAi constructs. The partial cDNA fragments were synthesized and cloned into appropriate dsRNAi expression vectors using convergent promoters to express the sense and antisense cDNA sequences, similar to the approach used above for PDS gene sequences and shown in Table 4 below.

TABLE 4

| | Selectable | Selectable | | DsRNA | |
|---|---|---|---|---|---|
| | marker | marker | | convergent | Fungal pathogen |
| Construct | promoter | gene | Terminator | promoters | cDNA fragments |
| PTS251 | GmPpsbA | aadA | NtTpsbA | GmPrrnPEP | FviDCL1/DCL2 |
| | | | | | dsRNA |
| PTS252 | GmPpsbA | aadA | NtTpsbA | GmPrrnPEP | FviAGO1/AGO2 |
| | | | | | dsRNA |
| PTS253 | GmPpsbA | aadA | NtTpsbA | GmPrrnPEP | SscAGL4/AGL2 |
| | | | | | dsRNA |
| PTS254 | GmPpsbA | aadA | NtTpsbA | GmPrrnPEP | SscDCL1/DCL2 |
| | | | | | dsRNA |

The plastid RNAi genes are cloned next to a selectable marker gene used for plastid transformation, and flanked on both sides by regions of identity to the soybean chloroplast genome to catalyze homologous recombination, as shown in the Figure below. All plastid transformation is via gene targeting into the plastid genome, resulting in a perfect copy of the trait gene(s) into the plastid genome in a predictable and predefined location.

Embryo axis explants from soybean cultivar Bert are used for plastid transformation, using methods similar to those described in US2016/0264983A1 except that embryo axis from fresh seeds were used. Chloroplast transformation events were selected on spectinomycin antibiotic medium, maintained in tissue culture for several months on selective media and subsequently confirmed by PCR and Southern blot analysis to be homoplasmic. Homoplasmic plants were rooted and transferred to the greenhouse to set seed prior to efficacy testing.

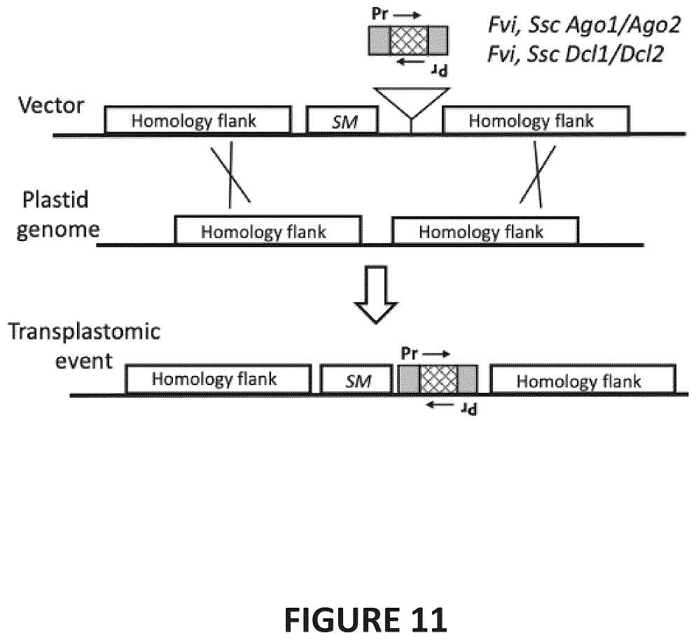

As illustrated in FIG. 11, Ago1/2 and Dcl1/2 fused cDNA sequences from F. virguliforme (Fvi) and S. sclerotiorum (Ssc) are expressed from convergent plastid promoters (Pr) to create dsRNA constructs. Selection of plastid transformants is via a selectable marker (SM) cloned next to the dsRNA transgene and surrounding by cloned plastid DNA regions to direct homologous recombination and insertion into the plastid genome.

Small RNA sequencing of plastid transformed lines was performed to characterize siRNAs derived from the plastid dsRNA transgene. Leaf tissue from 3 independent transplastomic plant clones were collected along with 3 independent wild-type untransformed plants as control. Small RNA libraries and sequencing was performed according to methods described above.

### Example: Treatments to Enhance Escape of dsRNA to the Cytoplasm

Certain chemical or biological agents that affect the integrity of the plastid membrane may enhance escape of nucleic acids or proteins to the cytoplasm and could subsequently increase the efficiency or amount of plastid-derived small RNA production that, in turn, would increase the efficacy against certain pathogens. As described above (Kwon et al., 2013), paraquat herbicide or bacterial infection caused the release of plastid-transgenic protein to the cytoplasm. Likewise, treatment of plastid dsRNA transgenic plants with herbicides such as paraquat, glyphosate, bialophos, atrazine, DCMU (3-(3,4-dichlorophenyl)-1,1-dimethylurea), mesotrione and the like, chemicals like Triton X-100 or cadmium (Lee and Back, 2022), or antimicrobial agents such as triclosan methylarsenic, is known to affect plastid membranes and leak their contents to the cytoplasm (Yan et al., 2022; Ye et al., 2003). Non-chemical treatments that cause leakage of chloroplast contents, such as short-term chilling stress, salinity stress (Hameed et al. 2021) or other biotic treatments may also enhance escape of plastid contents to the cytoplasm. Treatment of plastid transgenic plants can be performed in tissue culture or via application of plants growing in soil, and requires titration of these agents to enable recovery of plants after treatment. Enhanced escape of dsRNA from plastids increases efficacy against the variety of pathogens and enhanced processes as described in Table 3.

### Example: Plastid Antisense-Mediated Nuclear Gene Silencing

The PTS39 antisense PDS line is green and did not induce bleaching or apparent PDS silencing. This line does accumulate plastid PDS antisense RNA at easily detectable levels via northern blot (FIG. 5). One of the PTS39 lines (PTS39-3) accumulated detectable small RNA reads (˜20 RPM; Table 1) with a weak phasing score (17.3; Table 1), though to a much lower degree than observed in the PTS40 lines. This result suggests the PTS39 line accumulates an amount of small RNA that is below the threshold required to catalyze a gene silencing response under the conditions tested. This further suggests possible differences in efficiency of escape or stability of the antisense RNA in the cytoplasm, which may enable enhanced efficacy of antisense RNAs in gene silencing in future work.

Although the present disclosure has been described with reference to preferred embodiments, workers skilled in the art will recognize that changes may be made in form and detail without departing from the spirit and scope of the disclosure.

1. A method of introducing a DNA construct into a plant plastid genome to silence an endogenous nuclear-encoded gene to impart a beneficial trait to the plant.

2. The method of claim 1 and wherein the expression of a plastid-encoded double-stranded RNA silences the endogenous nuclear-encoded gene.

3. The method of claim 1 and further comprising introducing a plurality of DNA constructs into a plant plastid genome to silence one or more endogenous nuclear-encoded genes to impart one or more beneficial traits to the plant.

4. The method of claim 1, wherein the expression of a plastid-encoded sense-strand RNA construct silences the activity of an endogenous nuclear-encoded gene or wherein the expression of a plastid-encoded antisense-strand RNA construct silences the activity of an endogenous nuclear-encoded gene.

5. The method of claim 1 and further comprising using convergent plastid promoters or hairpin stem/loop dsRNA constructs for creating the plastid-encoded dsRNA that produces small RNAs.

6. The method of claim 1, wherein the DNA construct is a sense strand construct capable of producing small RNAs.

7. The method of claim 1, wherein the DNA construct is an anti-sense strand construct capable of producing small RNAs.

8. A method for controlling expression of a nuclear-encoded gene in a plant comprising expressing a plastid-encoded dsRNA that silences an endogenous nuclear-encoded gene in the plant to produce a transformed plant line expressing the selected trait.

9. The method of claim 8 and further comprising selecting a recipient plant cell, transforming the plant cell by introduction of a plastid transformation vector effecting expression of the plastid-encoded dsRNA capable of silencing the nuclear-encoded gene to the plant cell, and regenerating the plant from cells expressing the selected trait.

10. The transformed photosynthetic plant cell according to the method of claim 8 wherein the plant cell expresses the plastid-encoded dsRNA that silences the endogenous nuclear encoded gene wherein the plant cell exhibits the selected non-naturally occurring trait.

11. A method for controlling expression of a nuclear-encoded gene in a plant comprising expressing a plastid-encoded sense-strand RNA construct that silences activity of an endogenous nuclear-encoded gene in the plant to produce a transformed plant line expressing the selected trait.

12. The method of claim 11 and further comprising selecting a recipient plant cell, transforming the plant cell by introduction of a plastid transformation vector effecting expression of the plastid-encoded sense-strand RNA construct capable of silencing activity of a nuclear-encoded gene to the plant cell, and regenerating the plant from cells expressing the selected trait.

13. The transformed photosynthetic plant cell according to the method of claim 11 wherein the plant cell expresses the plastid-encoded sense-strand RNA construct that silences the activity of an endogenous nuclear encoded gene wherein the plant cell exhibits the selected non-naturally occurring trait.

14. A method of introducing a plurality of DNA constructs into a plant plastid genome to silence one or more endogenous nuclear-encoded genes, one or more pathogens, or a combination thereof to impart one or more beneficial traits to the plant.

15. A method for controlling a plant pathogen that is susceptible to small RNAs via expression of a plastid encoded dsRNA which silences an essential gene in the pathogen.

16. The method of claim 15 wherein the plastid-encoded dsRNA enables accumulation of small RNAs which enable transkingdom RNAi against the one or more pathogens.

17. A transformed photosynthetic plant cell having a plastid-encoded dsRNA that enables accumulation of small RNAs capable of silencing a nuclear-encoded gene for imparting a selected trait to the photosynthetic plant.

18. A method of liberating a nucleic acid or protein from a transgenic plant plastid to function in a non-plastid compartment of the cell.

19. A treatment that enhances the liberation or escape of a transgene-encoded RNA or protein from transgenic plastids, including treatment of the plants with a chemical herbicide, antimicrobial or chemical agent, or biotic stress conditions that affects plastid membranes

20. The treatment of claim 19 wherein treatment is preformed in tissue culture or via application to the plant growing in soil and further comprising titrating the chemical herbicide, antimicrobial or chemical agent to enable recovery of the plant after treatment.

21. A method for controlling expression of a nuclear-encoded gene in a plant comprising expressing a plastid-encoded antisense-strand RNA construct that silences activity of an endogenous nuclear-encoded gene in the plant to produce a transformed plant line expressing the selected trait.

22. The method of claim 21 and further comprising selecting a recipient plant cell, transforming the plant cell by introduction of a plastid transformation vector effecting expression of the plastid-encoded antisense-strand RNA construct capable of silencing activity of a nuclear-encoded gene to the plant cell, and regenerating the plant from cells expressing the selected trait.

23. A transformed photosynthetic plant cell expressing a plastid-encoded antisense-strand RNA construct that silences the activity of an endogenous nuclear encoded gene wherein the plant cell exhibits a selected non-naturally occurring trait.
